(19) United States
(12) Patent Application Publication
(10) Pub. No.: US 2014/0309109 A1
(43) Pub. Date: Oct. 16, 2014

(54) N-Thio-anthranilamide compounds and their use as pesticides

(71) Applicant: BASF SE, Ludwigshafen (DE)

(72) Inventors: Karsten Koerber, Eppelheim (DE); Florian Kaiser, Mannheim (DE); Prashant Deshmukh, Mannheim (DE); Deborah L. Culbertson, Fuquay Varina, NC (US); Paul Neese, Apex, NC (US); Koshi Gunjima, Cary, NC (US)

(73) Assignee: BASF SE, Ludwigshafen (DE)

(21) Appl. No.: 14/365,785

(22) PCT Filed: Dec. 20, 2012
(86) PCT No.: PCT/EP2012/076390

§ 371 Date: Jun. 16, 2014

Related U.S. Application Data
(60) Provisional application No. 61/578,267, filed on Dec. 21, 2011.

(51) Int. Cl. A01N 43/56; C07D 401/04

(52) U.S. Cl. CPC A01N 43/56; C07D 401/04
USPC 504/100; 546/275.4; 514/341

(57) ABSTRACT

The present invention relates to N-thio-anthranilamide compounds of the formula (I), the stereoisomers, the salts, the tautomers and the N-oxides thereof, wherein R¹ is hydrogen, C₁-C₆-alkyl or C₃-C₈-cycloalkyl; R² is hydrogen, halogen or cyano; R³ is hydrogen, C₁-C₆-alkyl, C₁-C₆-haloalkyl, C₂-C₆-alkenyl or the like; R⁴ is halogen or C₁-C₆-haloalkyl; R⁵ is an optionally substituted C₁-C₁₀-alkyl, C₃-C₈-cycloalkyl, C₂-C₁₀-alkenyl, C₂-C₁₀-alkynyl, phenyl, heterocyclic ring or the like; L is an optionally substituted C₁-C₈-alkanediyl, C₂-C₈-alkenediyl, C₂-C₈-alkynediyl, C₃-C₈-cycloalkanediyl or the like; G is an optionally substituted C₃-C₈-cycloalkyl, phenyl, heterocyclic ring or the like; and k is 0 or 1. The present invention further relates to a method for combating or controlling invertebrate pests, to a method for protecting plant propagation material and/or the plants which grow therefrom, to plant propagation material comprising at least one compound according to the present invention, to a method for treating or protecting an animal from infestation or infection by parasites, to a process for the preparation of a composition for treating infested or infected animals and/or for protecting animals against infestation or infection by parasites, and to a compound according to the invention for use as a medicament.

The present invention relates to N-thio-anthranilamide compounds and the stereoisomers, salts, tautomers and N-oxides thereof and to compositions comprising the same. The invention also relates to the use of the N-thio-anthranilamide compounds or of the compositions comprising such compounds for combating invertebrate pests. Furthermore, the invention relates to methods of applying such compounds.

Invertebrate pests and in particular insects, arthropods and nematodes destroy growing and harvested crops and attack wooden dwelling and commercial structures, thereby causing large economic loss to the food supply and to property. While a large number of pesticidal agents are known, due to the ability of target pests to develop resistance to said agents, there is an ongoing need for new agents for combating invertebrate pests such as insects, arachnids and nematodes. It is therefore an object of the present invention to provide compounds having a good pesticidal activity and showing a broad activity spectrum against a large number of different invertebrate pests, especially against difficult to control insects, arachnids and nematodes.

Anthranilamide compounds have been described in a number of patent applications (e.g. WO 01/70671, WO 03/015518, WO 03/024222, WO 2006/000336, WO 2006/068669, WO 2007/043677, WO 2008/130021, WO 03/015519, WO 2004/046129). WO 03/016300 describes a generic anthranilamide formula encompassing N-thio-anthranilamide compounds. WO 03/016284 describes inter alia certain N-thio-anthranilamide compounds. WO 2007/006670 describes N-thio-anthranilamide compounds with a sulfilimine or sulfoximine group and their use as pesticides. The unpublished application U.S. 61/522,752 discloses a process for preparing N-thio-anthranilamide compounds and derivates thereof obtained by introducing a substituted pyrazol-3-carbonyl moiety at the amino function of the phenyl ring. Such derivatives with a 2-pyridin-2-yl group and either a 5-trifluoromethyl or a 5-difluoromethyl group on the pyrazole ring as well as their use as pesticides are described in the unpublished applications U.S. 61/522,721 and U.S. 61/522,727.

It is an object of the present invention to provide further compounds having a high pesticidal activity against invertebrate pests, in particular against insect pest. The compounds should show a broad activity spectrum against a large number of different invertebrate pests, in particular against difficult to control insects, arachnids and nematodes.

It has been found that the above objectives can be achieved by N-thio-anthranilamide compounds of the general formula (I), as defined below, including their stereoisomers, their salts, in particular their agriculturally or veterinarily acceptable salts, their tautomers and their N-oxides.

Therefore, in a first aspect the present invention relates to compounds of formula (I),

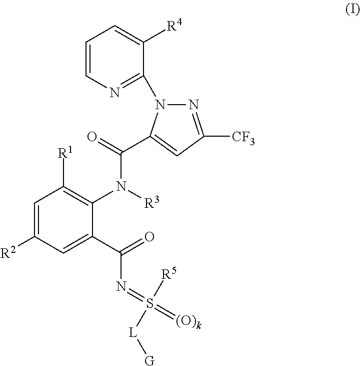

wherein

R¹ is selected from the group consisting of hydrogen, C₁-C₆-alkyl and C₃-C₈-cycloalkyl;

R² is selected from the group consisting of hydrogen, halogen and cyano;

R³ is selected from the group consisting of hydrogen, C₁-C₆-alkyl, C₁-C₆-haloalkyl, C₂-C₆-alkenyl, C₂-C₆-haloalkenyl, C₂-C₆-alkinyl, C₂-C₆-haloalkinyl, C₃-C₈-cycloalkyl, C₃-C₈-halocycloalkyl, C₁-C₄-alkoxy-C₁-C₄-alkyl, C₁-C₄-haloalkoxy-C₁-C₄-alkyl, C(═O)R^{a}, C(═O)OR^{b} and C(═O)NR^{c}R^{d};

R⁴ is halogen or C₁-C₆-haloalkyl;

R⁵ is selected from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or

R⁵ is a 3-, 4-, 5-, 6- or 7-membered saturated, partially unsaturated or fully unsaturated heterocyclic ring containing 1, 2 or 3 heteroatoms or heteroatom groups selected from N, O, S, NO, SO and SO₂, as ring members, where the heterocyclic ring may be substituted by one or more radicals R^{f};

L is selected from the group consisting of C₁-C₈-alkanediyl, C₂-C₈-alkenediyl, C₂-C₈-alkynediyl and C₃-C₈-cycloalkanediyl, where one or more CH₂ groups of the aforementioned radicals may be replaced by a C═O group, and where the aliphatic and cycloaliphatic moieties of the aforementioned radicals may be unsubstituted, partially or fully halogenated and/or may carry 1 or 2 substituents selected from the group consisting of C₁-C₄ alkoxy, C₁-C₄ alkyl and C₁-C₄ haloalkyl;

G is selected from the group consisting of C₃-C₈-cycloalkyl, which is unsubstituted or carries 1 to 10 substituents R^{e}, phenyl, which is unsubstituted or carries 1 to 5 substituents R^{f}, and a 3-, 4-, 5-, 6- or 7-membered saturated, partially unsaturated or fully unsaturated heterocyclic ring containing 1, 2 or 3 heteroatoms or heteroatom groups selected from N, O, S, NO, SO and SO₂, as ring members, where the heterocyclic ring may be substituted by one or more radicals R^{f};

R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,

R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy;

phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;

R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy; C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or

R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;

R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;

C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);

R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;

C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};

k is 0 or 1;

n is 0, 1 or 2;

or a stereoisomer, salt, tautomer or N-oxide thereof.

Furthermore, the invention relates to processes for the synthesis of compounds according to the invention and to intermediate compounds for the synthesis of compounds of formula (I).

The compounds of the present invention, i.e. the compounds of formula (I), their stereoisomers, their salts, their tautomers or their N-oxides, are particularly useful for controlling invertebrate pests, in particular for controlling arthropods and nematodes and especially insects. Therefore, the invention also relates to the use of a compound of the present invention, for combating or controlling invertebrate pests, in particular invertebrate pests of the group of insects, arachnids or nematodes.

The invention also relates to a composition comprising at least one compound according to the invention, including a stereoisomer, salt, tautomer or N-oxide thereof, and at least one inert liquid and/or solid carrier. In particular, the invention relates to an agricultural or veterinary composition comprising at least one compound according to the invention including a stereoisomer, an agriculturally or veterinarily acceptable salt, tautomer or an N-oxide thereof, and at least one liquid and/or solid carrier.

The present invention also relates to a method for combating or controlling invertebrate pests, especially invertebrate pests of the group of insects, arachnids or nematodes, which method comprises contacting said pest or its food supply, habitat or breeding grounds with a pesticidally effective amount of at least one compound according to the invention including a stereoisomer, salt, tautomer or N-oxide thereof or a composition according to the invention.

The present invention also relates to a method for protecting growing plants from attack or infestation by invertebrate pests, especially invertebrate pests of the group of insects, arachnids or nematodes, which method comprises contacting a plant, or soil or water in which the plant is growing or may grow, with a pesticidally effective amount of at least one compound according to the invention including a stereoisomer, salt, tautomer or N-oxide thereof or a composition according to the invention.

The present invention also relates to a method for the protection of plant propagation material, preferably seeds, from soil insects and of the seedlings' roots and shoots from soil and foliar insects comprising contacting the seeds before sowing and/or after pregermination with at least one compound according to the invention including a stereoisomer, salt, tautomer or N-oxide thereof or a composition according to the invention.

The present invention also relates to plant propagation material, preferably seed, comprising a compound according to the invention including a stereoisomer, salt, tautomer or N-oxide thereof, preferably in an amount of from 0.1 g to 10 kg per 100 kg of the plant propagation material.

The present invention also relates to the use of a compound according to the invention including a stereoisomer, salt, tautomer or N-oxide thereof or a composition according to the invention for combating or controlling invertebrate pests of the group of insects, arachnids or nematodes.

The present invention also relates to the use of a compound according to the invention including a stereoisomer, salt or N-oxide thereof or a composition according to the invention for protecting growing plants from attack or infestation by invertebrate pests of the group of insects, arachnids or nematodes.

The present invention also relates to the use of a compound according to the invention including a stereoisomer, veterinarily acceptable salt, tautomer or N-oxide thereof or a composition according to the invention for combating or controlling invertebrate parasites in and on animals.

The present invention also relates to a method for treating a non-human animal infested or infected by parasites or for preventing a non-human animal from getting infested or infected by parasites or for protecting a non-human animal against infestation or infection by parasites which comprises orally, topically or parenterally administering or applying to the non-human animal a parasiticidally effective amount of a compound according to the invention including a stereoisomer, veterinarily acceptable salt, tautomer or N-oxide thereof or a composition according to the invention.

The present invention also relates to the use of a compound according to the invention including a stereoisomer, veterinarily acceptable salt or N-oxide thereof or a composition according to the invention for the manufacture of a medicament for protecting an animal against infestation or infection by parasites or treating an animal infested or infected by parasites.

The present invention also relates to a process for the preparation of a composition for treating animals infested or infected by parasites, for preventing animals of getting infected or infested by parasites or protecting animals against infestation or infection by parasites which comprises a compound according to the invention including a stereoisomer, veterinarily acceptable salt, tautomer or N-oxide thereof.

The present invention also relates to a compound according to the invention including a stereoisomer, veterinarily acceptable salt, tautomer or N-oxide thereof for use as a medicament.

The present invention also relates to a compound according to the invention including a stereoisomer, veterinarily acceptable salt, tautomer or N-oxide thereof for use in the treatment, control, prevention or protection of animals against infestation or infection by parasites.

Depending on the substitution pattern, the compounds of the formula (I) may have one or more centers of chirality, in which case they are present as mixtures of enantiomers or diastereomers. The invention provides both the pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond, nitrogen-sulfur double bond or amide group. The term “stereoisomer(s)” encompasses both optical isomers, such as enantiomers or diastereomers, the latter existing due to more than one center of chirality in the molecule, as well as geometrical isomers (cis/trans isomers).

Depending on the substitution pattern, the compounds of the formula (I) may be present in the form of their tautomers. Hence the invention also relates to the tautomers of the formula (I) and the stereoisomers, salts, tautomers and N-oxides of said tautomers.

The term “N-oxide” includes any compound of the present invention which has at least one tertiary nitrogen atom that is oxidized to an N-oxide moiety. N-oxides of compounds (I) can in particular be prepared by oxidizing the ring nitrogen atom(s) of the pyridine ring and/or the pyrazole ring with a suitable oxidizing agent, such as peroxo carboxylic acids or other peroxides.

The compounds of the present invention may be amorphous or may exist in one ore more different crystalline states (polymorphs) which may have different macroscopic properties such as stability or show different biological properties such as activities. The present invention includes both amorphous and crystalline compounds of formula (I), their enantiomers or diastereomers, mixtures of different crystalline states of the respective compound of formula (I), its enantiomers or diastereomers, as well as amorphous or crystalline salts thereof.

Salts of the compounds of the present invention are preferably agriculturally and veterinarily acceptable salts. They can be formed in a customary method, e.g. by reacting the compound with an acid if the compound of the present invention has a basic functionality or by reacting the compound with a suitable base if the compound of the present invention has an acidic functionality.

Suitable agriculturally acceptable salts are especially the salts of those cations or the acid addition salts of those acids whose cations and anions, respectively, do not have any adverse effect on the pesticidal action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds of the present invention with an acid of the corresponding anion, preferably with hydrochloric acid, hydrobromic acid, sulfuric acid, phosphoric acid or nitric acid.

Veterinarily acceptable salts of the compounds of the present invention encompass the salts of those cations or the acid addition salts which are known and accepted in the art for the formation of salts for veterinary use. Suitable acid addition salts, e.g. formed by compounds of the present invention containing a basic nitrogen atom, e.g. an amino group, include salts with inorganic acids, for example hydrochlorides, sulfates, phosphates, and nitrates and salts of organic acids for example acetic acid, maleic acid, e.g. the monoacid salts or diacid salts of maleic acid, dimaleic acid, fumaric acid, e.g. the monoacid salts or diacid salts of fumaric acid, difumaric acid, methane sulfenic acid, methane sulfonic acid, and succinic acid.

The term “invertebrate pest” as used herein encompasses animal populations, such as arthropode pests, including insects and arachnids, as well as nematodes, which may attack plants thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “plants” comprises any types of plants including “non-cultivated plants” and in particular “cultivated plants”.

The term “non-cultivated plants” refers to any wild type species or related species or related genera of a cultivated plant.

The term “cultivated plants” is to be understood as including plants which have been modified by breeding, mutagenesis or genetic engineering. Genetically modified plants are plants, the genetic material of which has been modified by the use of recombinant DNA techniques so that under natural circumstances it cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s) (oligo- or polypeptides), e.g. by glycosylation or polymer additions such as prenylated, acetylated or farnesylated moieties or PEG moieties (e.g. as disclosed in Biotechnol Prog. 2001 July-August; 17(4):720-8., Protein Eng Des Sel. 2004 January; 17(1):57-66, Nat Protoc. 2007; 2(5): 1225-35., Curr Opin Chem Biol. 2006 October; 10(5):487-91. Epub 2006 Aug. 28., Biomaterials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e.g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e.g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e.g. Amflora® potato).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine. A partially or fully halogenated radical is termed below also “halo-radical”. For example, partially or fully halogenated alkyl is also termed haloalkyl.

The term “alkyl” as used herein (and in the alkyl moieties of other groups comprising an alkyl group, e.g. alkoxy, alkylcarbonyl, alkylthio, alkylsulfinyl, alkylsulfonyl and alkoxyalkyl) denotes in each case a straight-chain or branched alkyl group having usually from 1 to 10 carbon atoms, frequently from 1 to 6 carbon atoms, preferably 1 to 4 carbon atoms and in particular from 1 to 3 carbon atoms. Examples of C₁-C₄-alkyl are methyl, ethyl, n-propyl, isopropyl, n-butyl, 2-butyl (sec-butyl), isobutyl and tert-butyl. Examples for C₁-C₆-alkyl are, apart those mentioned for C₁-C₄-alkyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Examples for C₁-C₁₀-alkyl are, apart those mentioned for C₁-C₆-alkyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl, 2-propylpentyl, nonyl, decyl, 2-propylheptyl and 3-propylheptyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy, haloalkylthio, haloalkylcarbonyl, haloalkylsulfonyl and haloalkylsulfinyl) denotes in each case a straight-chain or branched alkyl group having usually from 1 to 10 carbon atoms (“C₁-C₁₀-haloalkyl”), frequently from 1 to 6 carbon atoms (“C₁-C₆-haloalkyl”), more frequently 1 to 4 carbon atoms (“C₁-C₁₀-haloalkyl”), wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl. Halomethyl is methyl in which 1, 2 or 3 of the hydrogen atoms are replaced by halogen atoms. Examples are bromomethyl, chloromethyl, dichloromethyl, trichloromethyl, fluoromethyl, difluoromethyl, trifluoromethyl, chlorofluoromethyl, dichlorofluoromethyl, chlorodifluoromethyl and the like. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. Examples for C₁-C₂-haloalkyl are, apart those mentioned for C₁-C₂-fluoroalkyl, chloromethyl, dichloromethyl, trichloromethyl, bromomethyl, chlorofluoromethyl, dichlorofluoromethyl, chlorodifluoromethyl, 1-chloroethyl, 2-chloroethyl, 2,2,-dichloroethyl, 2,2,2-trichloroethyl, 2-chloro-2-fluoroethyl, 2-chloro-2,2-difluoroethyl, 2,2-dichloro-2-fluoroethyl, 1-bromoethyl, and the like. Examples for C₁-C₄-haloalkyl are, apart those mentioned for C₁-C₂-haloalkyl, 1-fluoropropyl, 2-fluoropropyl, 3-fluoropropyl, 3,3-difluoropropyl, 3,3,3-trifluoropropyl, heptafluoropropyl, 1,1,1-trifluoroprop-2-yl, 3-chloropropyl, 4-chlorobutyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms (“C₃-C₁₀-cycloalkyl”), preferably 3 to 8 carbon atoms (“C₃-C₈-cycloalkyl”) or in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.1.1]hexyl, bicyclo[2.2.1]heptyl, bicyclo[3.1.1]heptyl, bicyclo[2.2.1]heptyl, bicyclo[2.2.2]octyl and bicyclo[3.2.1]octyl.

The term “cycloalkylene” (or cycloalkanediyl) as used herein in each case denotes an cycloalkyl radical as defined above, wherein one hydrogen atom at any position of the carbon backbone is replaced by one further binding site, thus forming a bivalent moiety.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, preferably 3 to 8 carbon atoms or in particular 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “cycloalkyl-alkyl” used herein denotes a cycloalkyl group, as defined above, which is bound to the remainder of the molecule via an alkylene group. The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkenyl” as used herein denotes in each case a monounsaturated straight-chain or branched hydrocarbon radical having usually 2 to 10 (“C₂-C₁₀-alkenyl”), preferably 2 to 6 carbon atoms (“C₂-C₆-alkenyl”), in particular 2 to 4 carbon atoms (“C₂-C₄-alkenyl”), and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “alkenylene” (or alkenediyl) as used herein in each case denotes an alkenyl radical as defined above, wherein one hydrogen atom at any position of the carbon backbone is replaced by one further binding site, thus forming a bivalent moiety.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) or 2 to 4 (“C₂-C₄-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 10 (“C₂-C₁₀-alkynyl”), frequently 2 to 6 (“C₂-C₆-alkynyl”), preferably 2 to 4 carbon atoms (“C₂-C₄-alkynyl”) and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term “alkynylene” (or alkynediyl) as used herein in each case denotes an alkynyl radical as defined above, wherein one hydrogen atom at any position of the carbon backbone is replaced by one further binding site, thus forming a bivalent moiety.

The term “haloalkynyl” as used herein, which is also expressed as “alkynyl which may be substituted by halogen”, refers to unsaturated straight-chain or branched hydrocarbon radicals having usually 3 to 10 carbon atoms (“C₂-C₁₀-haloalkynyl”), frequently 2 to 6 (“C₂-C₆-haloalkynyl”), preferably 2 to 4 carbon atoms (“C₂-C₄-haloalkynyl”), and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms (“C₁-C₁₀-alkoxy”), frequently from 1 to 6 carbon atoms (“C₁-C₆-alkoxy”), preferably 1 to 4 carbon atoms (“C₁-C₄-alkoxy”), which is bound to the remainder of the molecule via an oxygen atom. C₁-C₂-Alkoxy is methoxy or ethoxy. C₁-C₄-Alkoxy is additionally, for example, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “haloalkoxy” as used herein denotes in each case a straight-chain or branched alkoxy group, as defined above, having from 1 to 10 carbon atoms (“C₁-C₁₀-haloalkoxy”), frequently from 1 to 6 carbon atoms (“C₁-C₆-haloalkoxy”), preferably 1 to 4 carbon atoms (“C₁-C₄-haloalkoxy”), more preferably 1 to 3 carbon atoms (“C₁-C₃-haloalkoxy”), wherein the hydrogen atoms of this group are partially or totally replaced with halogen atoms, in particular fluorine atoms. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “alkoxyalkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. “C₁-C₆-Alkoxy-C₁-C₆-alkyl” is a C₁-C₆-alkyl group, as defined above, in which one hydrogen atom is replaced by a C₁-C₆-alkoxy group, as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “haloalkoxy-alkyl” as used herein denotes in each case alkyl as defined above, usually comprising 1 to 6 carbon atoms, preferably 1 to 4 carbon atoms, wherein 1 carbon atom carries an haloalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or alkyl-S—)” as used herein denotes in each case a straight-chain or branched saturated alkyl group as defined above, usually comprising 1 to 10 carbon atoms (“C₁-C₁₀-alkylthio”), frequently comprising 1 to 6 carbon atoms (“C₁-C₆-alkylthio”), preferably 1 to 4 carbon atoms (“C₁-C₄-alkylthio”), which is attached via a sulfur atom at any position in the alkyl group. C₁-C₂-Alkylthio is methylthio or ethylthio. C₁-C₄-Alkylthio is additionally, for example, n-propylthio, 1-methylethylthio (isopropylthio), butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “haloalkylthio” as used herein refers to an alkylthio group as defined above wherein the hydrogen atoms are partially or fully substituted by fluorine, chlorine, bromine and/or iodine. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₂-alkylsulfinyl” refers to a C₁-C₂-alkyl group, as defined above, attached via a sulfinyl [S(O)] group. The term “C₁-C₄-alkylsulfinyl” refers to a C₁-C₄-alkyl group, as defined above, attached via a sulfinyl [S(O)] group. The term “C₁-C₆-alkylsulfinyl” refers to a C₁-C₆-alkyl group, as defined above, attached via a sulfinyl [S(O)] group. C₁-C₂-alkylsulfinyl is methylsulfinyl or ethylsulfinyl. C₁-C₄-alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. The term “C₁-C₂-alkylsulfonyl” refers to a C₁-C₂-alkyl group, as defined above, attached via a sulfonyl [S(O)₂] group. The term “C₁-C₄-alkylsulfonyl” refers to a C₁-C₄-alkyl group, as defined above, attached via a sulfonyl [S(O)₂] group. The term “C₁-C₆-alkylsulfonyl” refers to a C₁-C₆-alkyl group, as defined above, attached via a sulfonyl [S(O)₂] group. C₁-C₂-alkylsulfonyl is methylsulfonyl or ethylsulfonyl. C₁-C₄-alkylsulfonyl is additionally, for example, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “alkylamino” as used herein denotes in each case a group —NHR, wherein R is a straight-chain or branched alkyl group usually having from 1 to 6 carbon atoms (“C₁-C₆-alkylamino”), preferably 1 to 4 carbon atoms (“C₁-C₄-alkylamino”). Examples of C₁-C₆-alkylamino are methylamino, ethylamino, n-propylamino, isopropylamino, n-butylamino, 2-butylamino, iso-butylamino, tert-butylamino, and the like.

The term “dialkylamino” as used herein denotes in each case a group-NRR′, wherein R and R′, independently of each other, are a straight-chain or branched alkyl group each usually having from 1 to 6 carbon atoms (“di-(C₁-C₆-alkyl)-amino”), preferably 1 to 4 carbon atoms (“di-(C₁-C₄-alkyl)-amino”). Examples of a di-(C₁-C₆-alkyl)-amino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The term “alkylaminosulfonyl” as used herein denotes in each case a straight-chain or branched alkylamino group as defined above, which is bound to the remainder of the molecule via a sulfonyl [S(O)₂] group. Examples of an alkylaminosulfonyl group are methylaminosulfonyl, ethylaminosulfonyl, n-propylaminosulfonyl, isopropylaminosulfonyl, n-butylaminosulfonyl, 2-butylaminosulfonyl, iso-butylaminosulfonyl, tert-butylaminosulfonyl, and the like.

The term “dialkylaminosulfonyl” as used herein denotes in each case a straight-chain or branched alkylamino group as defined above, which is bound to the remainder of the molecule via a sulfonyl [S(O)₂] group. Examples of an dialkylaminosulfonyl group are dimethylaminosulfonyl, diethylaminosulfonyl, dipropylaminosulfonyl, dibutylaminosulfonyl, methyl-ethyl-aminosulfonyl, methyl-propyl-aminosulfonyl, methyl-isopropylaminosulfonyl, methyl-butyl-aminosulfonyl, methyl-isobutyl-aminosulfonyl, ethyl-propyl-aminosulfonyl, ethyl-isopropylaminosulfonyl, ethyl-butyl-aminosulfonyl, ethyl-isobutyl-aminosulfonyl, and the like.

The suffix “-carbonyl” in a group denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl.

The term “het(ero)aryl” as used herein refers to a mono-, bi- or tricyclic heteroaromatic hydrocarbon radical, preferably to a monocyclic heteroaromatic radical, such as pyridyl, pyrimidyl and the like.

The term “3-, 4-, 5-, 6- or 7-membered saturated, partially unsaturated or fully unsaturated heterocyclic ring containing 1, 2 or 3 heteroatoms or heteroatom groups selected from N, O, S, NO, SO and SO₂, as ring members” [wherein “fully unsaturated” also includes “aromatic”] as used herein denotes monocyclic radicals, the monocyclic radicals being saturated, partially unsaturated or fully unsaturated (including aromatic). Unsaturated rings contain at least one C—C and/or C—N and/or N—N double bond(s). Fully unsaturated rings contain as many conjugated C—C and/or C—N and/or N—N double bonds as allowed by the ring size. Fully unsaturated 5- or 6-membered heterocyclic rings are aromatic. The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

Examples of a 3-, 4-, 5-, 6- or 7-membered saturated heterocyclic ring include: oxiranyl, thiiranyl, aziridinyl, oxetanyl, thietanyl, azetidinyl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

A 3-, 4-, 5-, 6- or 7-membered fully unsaturated (including aromatic) heterocyclic ring is e.g. a 5- or 6-membered fully unsaturated (including aromatic) heterocyclic ring. Examples are: 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl.

When R^{c} and R^{d}, together with the nitrogen atom to which they are bound form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, this is an N-bound heterocyclic ring which apart the nitrogen ring atom may additionally contain 1, 2, 3 or 4 further heteroatoms or heteroatom groups selected from N, O, S, NO, SO and SO₂, as ring members. Examples are aziridin-1-yl, azetidin-1-yl, pyrrolidine-1-yl, pyrazolidin-1-yl, imidazolin-1-yl, oxazolidin-3-yl, isoxazolidin-3-yl, thiazolidin-1-yl, isothiazolidin-1-yl, triazolidin-1-yl, piperdon-1-yl, piperazine-1-yl, morpholin-4-yl, thiomorpholin-1-yl, 1,1-dioxothiomorpholin-4-yl, pyrrolin-1-yl, pyrrolin-1-yl, imidazolin-1-yl, dihydropyridin-1-yl, tetrahydropyridin-1-yl, pyrrol-1-yl, pyrazo-1-yl, imidazol-1-yl and the like.

The remarks made below as to preferred embodiments of the variables (substituents) of the compounds of formulae (I), (I-a) and (I-b) are valid on their own as well as preferably in combination with each other, as well as in combination with the stereoisomers, salts, tautomers or N-oxides thereof.

The remarks made below concerning preferred embodiments of the variables further are valid on their own as well as preferably in combination with each other concerning the compounds of formulae (I), (I-a) or (I-b), where applicable, as well as concerning the uses and methods according to the invention and the composition according to the invention.

Preferred compounds according to the invention are compounds of formulae (I), (I-a) or (I-b) or a stereoisomer, salt, tautomer or N-oxide thereof, wherein the salt is an agriculturally or veterinarily acceptable salt. Further preferred compounds according to the invention are compounds of formulae (I), (I-a) or (I-b) or a stereoisomer or salt thereof, especially an agriculturally or veterinarily acceptable salt. Particularly preferred compounds according to the invention are compounds of formulae (I), (I-a) or (I-b) or a salt thereof, especially an agriculturally or veterinarily acceptable salt thereof.

Preferred are compounds of formula (I), wherein R¹ is selected from hydrogen, C₁-C₄-alkyl and C₃-C₆-cycloalkyl, more preferably from C₁-C₄-alkyl and C₃-C₆-cycloalkyl, in particular from methyl, ethyl, CH₂CH₂CH₃, CH(CH₃)₂, CH₂CH₂CH₂CH₃, CH(CH₃)CH₂CH₃, CH₂CH(CH₃)CH₃, cyclopropyl, cyclobutyl, cyclopentyl and cyclohexyl, especially from methyl, ethyl, CH(CH₃)₂, cyclopropyl, cyclobutyl, cyclopentyl and cyclohexyl, specifically from methyl and ethyl, and more specifically is methyl.

Preferred are compounds of formula (I), wherein R² is selected from F, Cl, Br, I and CN, in particular from F, Cl, Br and CN, especially from Cl, Br and CN, and specifically from Cl and Br.

Preferred are compounds of formula (I), wherein R³ is selected from hydrogen, C₁-C₄-alkyl, C₁-C₄-haloalkyl, C₂-C₆-alkenyl, C₂-C₆-haloalkenyl, C₃-C₆-cycloalkyl, C₃-C₆-halocycloalkyl, C₁-C₂-alkoxy-C₁-C₂-alkyl, C₁-C₂-haloalkoxy-C₁-C₂-alkyl, C(═O)R^{a}, C(═O)OR^{b} and C(═O)NR^{c}R^{d}.

In particular, R³ is selected from hydrogen, C₁-C₂-alkyl and C₁-C₂ haloalkyl, especially from hydrogen, methyl and halomethyl, and specifically is hydrogen.

Preferred are compounds of formula (I), wherein R⁴ is selected from F, Cl and Br, especially from Cl and Br; and specifically R⁴ is Cl.

Preferred are compounds of formula (I), wherein R⁵ is selected from C₁-C₆-alkyl, C₃-C₆-cycloalkyl, C₂-C₆-alkenyl, C₂-C₆-alkynyl, wherein the aforementioned radicals may be substituted with 1 to 10 substituents R^{e}, and phenyl, which is unsubstituted or carries 1 to 4 radicals R^{f}, or R⁵ is a 4-, 5-, 6- or 7-membered saturated, partially unsaturated or fully unsaturated heterocyclic ring containing 1, 2 or 3 heteroatoms selected from N, O and S, as ring members, where the heterocyclic ring may be substituted by 1 to 4 radicals R^{f}.

More preferred are compounds of formula (I), wherein R⁵ is selected from C₁-C₆-alkyl, C₃-C₆-cycloalkyl, C₂-C₆-alkenyl, C₂-C₆-alkynyl, wherein the aforementioned radicals may be substituted with 1 to 6 substituents R^{e}, and phenyl, which is unsubstituted or carries 1 to 4 radicals R^{f}, or R⁵ is a 5-, 6- or 7-membered saturated, partially unsaturated or fully unsaturated heterocyclic ring containing 1, 2 or 3 heteroatoms selected from N, O and S, as ring members, where the heterocyclic ring may be substituted by 1, 2 or 3 radicals selected from halogen, cyano, C₁-C₄-alkyl, C₁-C₄-alkoxy and C₁-C₄-haloalkyl.

In particular, R⁵ is selected from C₁-C₆-alkyl, C₃-C₆-cycloalkyl, C₂-C₆-alkenyl, C₂-C₆-alkynyl, wherein the aforementioned radicals may be substituted with 1 to 4 substituents selected from halogen, cyano, C₁-C₆-alkyl and C₃-C₆-cycloalkyl, and phenyl, which is unsubstituted or carries 1, 2 or 3 radical selected from halogen, cyano, methyl, methoxy, trifluoromethyl and difluoromethyl.

Especially, R⁵ is selected from C₁-C₆-alkyl, C₃-C₆-cycloalkyl, C₂-C₆-alkenyl, C₂-C₆-alkynyl, wherein the aforementioned radicals may be substituted with 1 or 2 substituents selected from F, Cl, Br, cyano, C₁-C₄-alkyl and C₃-C₆-cycloalkyl, and phenyl, which is unsubstituted or carries 1 or 2 radical selected from Cl, Br, cyano, methyl, methoxy, trifluoromethyl and difluoromethyl.

Specifically, R⁵ is selected from CH₃, CH₂CH₃, CH═CH₂, CH₂CH₂CH₃, CH(CH₃)₂, CH₂CH₂CH₂CH₃, C(CH₃)₃, CH₂CH(CH₃)₂, CH(CH₃)CH₂CH₃, CH₂CH═CH₂, CH₂C═CH, CH(CH₃)CH═CH₂, CHF₂, CH₂Cl, CH₂CH₂CN, CH₂CH₂Cl, cyclopropyl, cyclobutyl, cyclopentyl, cyclohexyl, cyclopropylmethyl, 1-cyclopropylethyl, cyclopentylmethyl, cyclohexylmethyl and phenyl; and in particular is CH₃, CH₂CH₃, CH(CH₃)₂ or cyclopropylmethyl.

Preferred are compounds of formula (I), wherein L is selected from the group consisting of C₁-C₆-alkanediyl, C₂-C₆-alkenediyl, C₂-C₆-alkynediyl and C₃-C₇-cycloalkanediyl, where the aliphatic and cycloaliphatic moieties of the aforementioned radicals may be unsubstituted, partially or fully halogenated and/or may carry 1 or 2 substituents selected from the group consisting of C₁-C₄-alkoxy, C₁-C₄-alkyl and C₁-C₄-haloalkyl;

In particular, L is selected from C₁-C₆-alkanediyl, C₂-C₆-alkenediyl and C₃-C₇-cycloalkanediyl, where the aforementioned radicals may be unsubstituted, partially or fully halogenated and/or may carry 1 or 2 substituents selected from the group consisting of C₁-C₃-alkoxy, C₁-C₃-alkyl and C₁-C₃-haloalkyl.

Especially, L is selected from the group consisting of C₁-C₆-alkandiyl and C₂-C₆-alkendiyl, where the aforementioned radicals may be unsubstituted, partially or fully halogenated.

Specifically, L is selected from the group consisting of CH₂, CH₂CH₂, CH(CH₃), CH═CH, CH₂CH₂CH₂, C(CH₃)₂, CH₂CH₂CH₂CH₂, CH₂C(CH₃)₂, CH(CH₃)CH₂CH₂, CH₂CH═CH, C(CH₃)CH═CH₂, CF₂, CHCl and CH₂CHCl, especially is CH₂, CH₂CH₂, CH(CH₃) or C(CH₃)₂, and in particular is CH₂ or CH(CH₃).

Preferred are compounds of formula (I), wherein G is selected from the group consisting of C₃-C₈-cycloalkyl, which is unsubstituted or carries 1 to 8 substituents R^{e}, phenyl, which is unsubstituted or carries 1 to 4 substituents R^{f}, and a 4-, 5-, 6- or 7-membered saturated, partially unsaturated or fully unsaturated heterocyclic ring containing 1, 2 or 3 heteroatoms or heteroatom groups selected from N, O, S, NO, SO and SO₂, as ring members, where the heterocyclic ring may be substituted by 1 to 4 radicals R^{f}

In particular, G is selected from C₃-C₇-cycloalkyl, which is unsubstituted or carries 1 to 4 substituents selected from halogen, cyano, C₁-C₆-alkyl and C₂-C₆-alkenyl, phenyl and a 4-, 5-, 6- or 7-membered saturated, partially unsaturated or fully unsaturated heterocyclic ring containing 1, 2 or 3 heteroatoms selected from N, O and S, as ring members, wherein each of the last two radicals mentioned is unsubstituted or carries 1, 2 or 3 radicals selected from halogen, cyano, C₁-C₄-alkyl, C₁-C₄-alkoxy and C₁-C₄-haloalkyl.

Especially, G is selected from C₃-C₇-cycloalkyl, which is unsubstituted or carries 1 to 4 substituents selected from halogen, cyano and C₁-C₄-alkyl and phenyl, which is unsubstituted or carries 1, 2 or 3 radicals selected from halogen, cyano and C₁-C₄-alkyl.

Specifically, G is C₃-C₇-cycloalkyl, especially cyclopropyl, cyclobutyl, cyclopentyl or cyclohexyl, and in particular is cyclopropyl, cyclopentyl or cyclohexyl.

Preferred are compounds of formula (I), wherein k is 0.

In this context, the variables R^{a}, R^{b}, R^{c}, R^{d}, R^{e}, R^{f} and n, independently of each other, preferably have one of the following meanings:

R^{a} is selected from C₁-C₆-alkyl, C₂-C₆-alkenyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy; phenyl, benzyl and pyridyl, wherein the last three radicals may be unsubstituted, partially or fully halogenated and/or carry 1 or 2 substituents selected from C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylamino and di-(C₁-C₄-alkyl)amino.

More preferably R^{a} is selected from C₁-C₄-alkyl, C₂-C₄-alkenyl, wherein the aforementioned radicals may be unsubstituted, partially or fully halogenated and/or may carry 1 or 2 substituents selected from C₁-C₂ alkoxy; phenyl and benzyl, wherein the last two radicals may be unsubstituted, partially or fully halogenated and/or carry 1 or 2 substituents selected from C₁-C₂-alkyl, C₁-C₂-haloalkyl, C₁-C₂-alkoxy and C₁-C₂-haloalkoxy; and in particular selected from C₁-C₄-alkyl, C₁-C₄-haloalkyl and benzyl which may be unsubstituted, partially or fully halogenated and/or carry 1 or 2 substituents selected from methyl, halomethyl, methoxy and halomethoxy.

R^{b} is selected from C₁-C₆-alkyl, C₂-C₆-alkenyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy; phenyl, benzyl and pyridyl, wherein the last three radicals may be unsubstituted, partially or fully halogenated and/or carry 1 or 2 substituents selected from C₁-C₄-alkyl, C₁-C₄-haloalkyl, C₁-C₄-alkoxy, C₁-C₄-haloalkoxy and (C₁-C₄-alkoxy)carbonyl.

More preferably R^{b} is selected from C₁-C₄-alkyl, C₂-C₄-alkenyl, wherein the aforementioned radicals may be unsubstituted, partially or fully halogenated and/or may carry 1 or 2 substituents selected from C₁-C₂-alkoxy; phenyl and benzyl, wherein the last two radicals may be unsubstituted, partially or fully halogenated and/or carry 1 or 2 substituents selected from C₁-C₂-alkyl, C₁-C₂-haloalkyl, C₁-C₂-alkoxy and C₁-C₂-haloalkoxy, and in particular selected from C₁-C₄-alkyl, C₁-C₄-haloalkyl and benzyl which may be unsubstituted, partially or fully halogenated and/or carry 1 or 2 substituents selected from methyl, halomethyl, methoxy and halomethoxy.

R^{c}, R^{d} are, independently from one another and independently of each occurrence, selected from hydrogen, C₁-C₆-alkyl, C₂-C₆-alkenyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy; C₁-C₄-alkylsulfonyl, phenyl and benzyl, wherein the two last mentioned radicals may be unsubstituted, partially or fully halogenated and/or carry 1 or 2 substituents selected from C₁-C₄-alkyl, C₁-C₄-haloalkyl, C₁-C₄-alkoxy and C₁-C₄ haloalkoxy; or R^{c} and R^{d}, together with the nitrogen atom to which they are bound, may form a 5-, 6- or 7-membered saturated, partially unsaturated or fully unsaturated heterocyclic ring which may additionally contain 1 or 2 further heteroatoms or heteroatom groups selected from N, O and S, as ring members, where the heterocylic ring may optionally be substituted with halogen, C₁-C₂-haloalkyl, C₁-C₂-alkoxy or C₁-C₂-haloalkoxy.

More preferably R^{c}, R^{d} are, independently from one another and independently of each occurrence, selected from hydrogen, C₁-C₄-alkyl, C₁-C₄-haloalkyl and benzyl, or R^{c} and R^{d}, together with the nitrogen atom to which they are bound, may form a 5- or 6-membered saturated or partly unsaturated heterocyclic ring. In particular, R^{c}, R^{d} are, independently from one another and independently of each occurrence, hydrogen, C₁-C₃-alkyl, C₁-C₂-haloalkyl, benzyl, or together with the nitrogen atom to which they are bound form a pyrrolidine or a piperidine ring.

R^{e} is selected from halogen, cyano, nitro, —OH, C₁-C₄-alkyl, C₂-C₄-alkenyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy; C₁-C₆-alkoxy, C₁-C₆-haloalkoxy, C₁-C₆-alkylthio, C₁-C₆-alkylsulfonyl, —NR^{c}R^{d}, —C(═O)R^{a}, —C(═O)OR^{b}, phenyl, benzyl and phenoxy, wherein the last three radicals may be unsubstituted, partially or fully halogenated and/or carry 1 or 2 substituents selected from C₁-C₄-alkyl, C₁-C₄-haloalkyl, C₁-C₄-alkoxy and C₁-C₄-haloalkoxy.

More preferably R^{e} is selected from F, Cl, Br, cyano, nitro, —OH, C₁-C₄-alkyl, C₂-C₄-alkenyl, C₃-C₈-cycloalkyl, wherein the aliphatic and cycloaliphatic moieties of the aforementioned radicals may be unsubstituted, partially or fully halogenated and/or may carry 1 or 2 radicals selected from C₁-C₂-alkoxy; C₁-C₄-alkoxy, C₁-C₄-haloalkoxy, —NR^{c}R^{d}, —C(═O)R^{a}, phenyl and benzyl, wherein the last two radicals may be unsubstituted, partially or fully halogenated and/or carry 1 or 2 substituents selected from C₁-C₂-alkyl, C₁-C₂-haloalkyl, C₁-C₂-alkoxy and C₁-C₂-haloalkoxy; and in particular from F, Cl, —OH, C₁-C₄-alkyl, C₁-C₄-haloalkyl, C₁-C₄-alkoxy, C₁-C₄-haloalkoxy, phenyl and benzyl, wherein the last two radicals may carry 1 or 2 substituents selected F, Cl, methyl, halomethyl, methoxy and halomethoxy.

R^{f} is selected from halogen, cyano, nitro, —OH, C₁-C₆-alkyl, C₂-C₆-alkenyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₂ alkoxy; C₁-C₄-alkoxy, C₁-C₄-haloalkoxy, —OR^{a}, —NR^{c}R^{d}, —S(O)ₙR^{a}, —C(═O)R^{a} and —C(═O)OR^{b}.

More preferably R^{f} is selected from F, Cl, Br, nitro, —OH, C₁-C₄-alkyl, C₂-C₄-alkenyl, wherein the aliphatic moieties of the aforementioned radicals may be unsubstituted, partially or fully halogenated and/or may carry 1 or 2 radicals selected from C₁-C₂ alkoxy; C₁-C₄-alkoxy, C₁-C₄-haloalkoxy, —OR^{a}, —NR^{c}R^{d} and C(═O)R^{a}; and in particular from F, Cl, nitro, C₁-C₄-alkyl, C₁-C₄-haloalkyl, C₁-C₂-alkoxy and C₁-C₂-haloalkoxy.

n is 1 or 2, wherein, in the case of several occurrences, n may be identical or different. More preferably n is 2.

In a preferred embodiment, the compound of formula (I) is of the general formula (I-a)

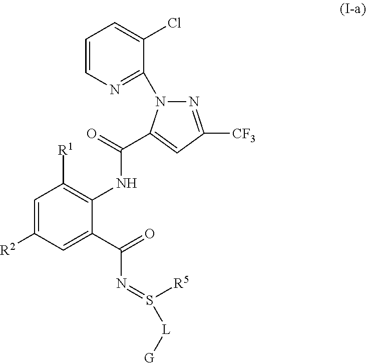

wherein

R¹, R², R⁵, L and G have one of the general meanings, or, in particular, one of the preferred meanings given above.

Preferred are compounds of formula (I-a), wherein
R¹ is selected from methyl, ethyl, CH₂CH₂CH₃, CH(CH₃)₂, CH₂CH₂CH₂CH₃, CH(CH₃)CH₂CH₃, CH₂CH(CH₃)CH₃, cyclopropyl, cyclobutyl, cyclopentyl and cyclohexyl, and in particular from methyl, ethyl, CH(CH₃)₂, cyclopropyl, cyclobutyl, cyclopentyl and cyclohexyl;
R² is selected from F, Cl, Br, I and CN, and in particular from F, Cl, Br and CN;
R⁵ is selected from C₁-C₆-alkyl, C₃-C₆-cycloalkyl, C₂-C₆-alkenyl, C₂-C₆-alkynyl, wherein the aforementioned radicals may be substituted with 1 to 6 substituents R^{e}, and phenyl, which is unsubstituted or carries 1 to 4 radicals R^{f}, or R⁵ is a 5-, 6- or 7-membered saturated, partially unsaturated or fully unsaturated heterocyclic ring containing 1, 2 or 3 heteroatoms selected from N, O and S, as ring members, where the heterocyclic ring may be substituted by 1, 2 or 3 radicals selected from halogen, cyano, C₁-C₄-alkyl, C₁-C₄-alkoxy and C₁-C₄-haloalkyl;
L is selected from C₁-C₆-alkanediyl, C₂-C₆-alkenediyl and C₃-C₇-cycloalkanediyl, where the aforementioned radicals may be unsubstituted, partially or fully halogenated and/or may carry 1 or 2 substituents selected from the group consisting of C₁-C₃-alkoxy, C₁-C₃-alkyl and C₁-C₃-haloalkyl;
G is selected from C₃-C₇-cycloalkyl, which is unsubstituted or carries 1 to 4 substituents selected from halogen, cyano, C₁-C₆-alkyl and C₂-C₆-alkenyl, phenyl and a 4-, 5-, 6- or 7-membered saturated, partially unsaturated or fully unsaturated heterocyclic ring containing 1, 2 or 3 heteroatoms selected from N, O and S, as ring members, wherein each of the last two radicals mentioned is unsubstituted or carries 1, 2 or 3 radicals selected from halogen, cyano, C₁-C₄-alkoxy and C₁-C₄-haloalkyl.

Particularly preferred are compounds of formula (I-a), wherein
R¹ is selected from methyl, ethyl, CH₂CH₂CH₃, CH(CH₃)₂, CH₂CH₂CH₂CH₃, cyclopropyl, cyclobutyl, cyclopentyl and cyclohexyl, and specifically from methyl, ethyl, CH(CH₃)₂, cyclopropyl, cyclobutyl, cyclopentyl and cyclohexyl;
R² is selected from F, Cl, Br and CN, specifically from Cl, Br and CN and more specifically from Cl and Br;
R⁵ is selected from C₁-C₆-alkyl, C₃-C₆-cycloalkyl, C₂-C₆-alkenyl, C₂-C₆-alkynyl, wherein the aforementioned radicals may be substituted with 1 or 2 substituents selected from F, Cl, Br, cyano, C₁-C₄-alkyl and C₃-C₆-cycloalkyl, and phenyl, which is unsubstituted or carries 1 or 2 radical selected from Cl, Br, cyano, methyl, methoxy, trifluoromethyl and difluoromethyl;
L is selected from the group consisting of C₁-C₆-alkandiyl and C₂-C₆-alkendiyl, where the aforementioned radicals may be unsubstituted, partially or fully halogenated;
G is selected from C₃-C₇-cycloalkyl, which is unsubstituted or carries 1 to 4 substituents selected from halogen, cyano and C₁-C₄-alkyl and phenyl, which is unsubstituted or carries 1, 2 or 3 radicals selected from halogen, cyano and C₁-C₄-alkyl.

Especially preferred are compounds of formula (I-a), wherein
R¹ is selected from methyl, ethyl, CH(CH₃)₂, cyclopropyl, cyclobutyl, cyclopentyl and cyclohexyl, and more specifically from methyl and ethyl;
R² is selected from Cl, Br and CN and more specifically from Cl and Br;
R⁵ is selected from CH₃, CH₂CH₃, CH═CH₂, CH₂CH₂CH₃, CH(CH₃)₂, CH₂CH₂CH₂CH₃, C(CH₃)₃, CH₂CH(CH₃)₂, CH(CH₃)CH₂CH₃, CH₂CH═CH₂, CH₂C≡CH, CH(CH₃)CH═CH₂, CHF₂, CH₂Cl, CH₂CH₂CN, CH₂CH₂Cl, cyclopropyl, cyclobutyl, cyclopentyl, cyclohexyl, cyclopropylmethyl, 1-cyclopropylethyl, cyclopentylmethyl, cyclohexylmethyl and phenyl;
L is selected from the group consisting of CH₂, CH₂CH₂, CH(CH₃), CH═CH, CH₂CH₂CH₂, C(CH₃)₂, CH₂CH₂CH₂CH₂, CH₂C(CH₃)₂, CH(CH₃)CH₂CH₂, CH₂CH═CH, C(CH₃)CH═CH₂, CF₂, CHCl and CH₂CHCl;
G is C₃-C₇-cycloalkyl, and in particular cyclopropyl, cyclobutyl, cyclopentyl or cyclohexyl

Specifically preferred are compounds of formula (I-a), wherein
R¹ is methyl or ethyl;
R² is Cl, Br or CN;
R⁵ is CH₃, CH₂CH₃, CH(CH₃)₂ or cyclopropylmethyl,
L is CH₂, CH₂CH₂, CH(CH₃) or C(CH₃)₂;
G is cyclopropyl, cyclobutyl, cyclopentyl or cyclohexyl.

More specifically preferred are compounds of formula (I-a), wherein
R¹ is methyl;
R² is Cl, Br or CN;
R⁵ is CH₃, CH₂CH₃, CH(CH₃)₂ or cyclopropylmethyl,
L is CH₂ or CH(CH₃);
G is cyclopropyl, cyclopentyl or cyclohexyl.

In another preferred embodiment, the compound of formula (I) is of the general formula (I-b)

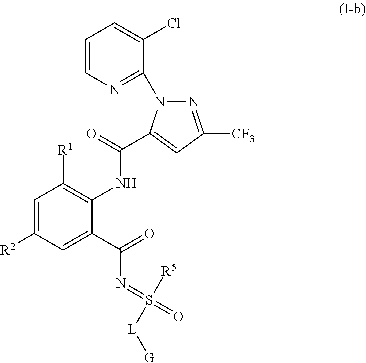

wherein

R¹, R², R⁵, L and G have one of the general meanings, or, in particular, one of the preferred meanings given above.

Preferred are compounds of formula (I-b), wherein
R¹ is selected from methyl, ethyl, CH₂CH₂CH₃, CH(CH₃)₂, CH₂CH₂CH₂CH₃, CH(CH₃)CH₂CH₃, CH₂CH(CH₃)CH₃, cyclopropyl, cyclobutyl, cyclopentyl and cyclohexyl, and in particular from methyl, ethyl, CH(CH₃)₂, cyclopropyl, cyclobutyl, cyclopentyl and cyclohexyl;
R² is selected from F, Cl, Br, I and CN, and in particular from F, Cl, Br and CN;
R⁵ is selected from C₁-C₆-alkyl, C₃-C₆-cycloalkyl, C₂-C₆-alkenyl, C₂-C₆-alkynyl, wherein the aforementioned radicals may be substituted with 1 to 6 substituents R^{e}, and phenyl, which is unsubstituted or carries 1 to 4 radicals R^{f}, or R⁵ is a 5-, 6- or 7-membered saturated, partially unsaturated or fully unsaturated heterocyclic ring containing 1, 2 or 3 heteroatoms selected from N, O and S, as ring members, where the heterocyclic ring may be substituted by 1, 2 or 3 radicals selected from halogen, cyano, C₁-C₄-alkoxy and C₁-C₄-haloalkyl;
L is selected from C₁-C₆-alkanediyl, C₂-C₆-alkenediyl and C₃-C₇-cycloalkanediyl, where the aforementioned radicals may be unsubstituted, partially or fully halogenated and/or may carry 1 or 2 substituents selected from the group consisting of C₁-C₃-alkoxy, C₁-C₃-alkyl and C₁-C₃-haloalkyl;
G is selected from C₃-C₇-cycloalkyl, which is unsubstituted or carries 1 to 4 substituents selected from halogen, cyano, C₁-C₆-alkyl and C₂-C₆-alkenyl, phenyl and a 4-, 5-, 6- or 7-membered saturated, partially unsaturated or fully unsaturated heterocyclic ring containing 1, 2 or 3 heteroatoms selected from N, O and S, as ring members, wherein each of the last two radicals mentioned is unsubstituted or carries 1, 2 or 3 radicals selected from halogen, cyano, C₁-C₄-alkoxy and C₁-C₄-haloalkyl.

Particularly preferred are compounds of formula (I-b), wherein
R¹ is selected from methyl, ethyl, CH₂CH₂CH₃, CH(CH₃)₂, CH₂CH₂CH₂CH₃, cyclopropyl, cyclobutyl, cyclopentyl and cyclohexyl, and specifically from methyl, ethyl, CH(CH₃)₂, cyclopropyl, cyclobutyl, cyclopentyl and cyclohexyl;
R² is selected from F, Cl, Br and CN, specifically from Cl, Br and CN and more specifically from Cl and Br;
R⁵ is selected from C₁-C₆-alkyl, C₃-C₆-cycloalkyl, C₂-C₆-alkenyl, C₂-C₆-alkynyl, wherein the aforementioned radicals may be substituted with 1 or 2 substituents selected from F, Cl, Br, cyano, C₁-C₄-alkyl and C₃-C₆-cycloalkyl, and phenyl, which is unsubstituted or carries 1 or 2 radical selected from Cl, Br, cyano, methyl, methoxy, trifluoromethyl and difluoromethyl;
L is selected from the group consisting of C₁-C₆-alkandiyl and C₂-C₆-alkendiyl, where the aforementioned radicals may be unsubstituted, partially or fully halogenated;
G is selected from C₃-C₇-cycloalkyl, which is unsubstituted or carries 1 to 4 substituents selected from halogen, cyano and C₁-C₄-alkyl and phenyl, which is unsubstituted or carries 1, 2 or 3 radicals selected from halogen, cyano and C₁-C₄-alkyl.

Especially preferred are compounds of formula (I-b), wherein
R¹ is selected from methyl, ethyl, CH(CH₃)₂, cyclopropyl, cyclobutyl, cyclopentyl and cyclohexyl, and more specifically from methyl and ethyl;
R² is selected from Cl, Br and CN and more specifically from Cl and Br;
R⁵ is selected from CH₃, CH₂CH₃, CH═CH₂, CH₂CH₂CH₃, CH(CH₃)₂, CH₂CH₂CH₂CH₃, C(CH₃)₃, CH₂CH(CH₃)₂, CH(CH₃)CH₂CH₃, CH₂CH═CH₂, CH₂C≡CH, CH(CH₃)CH═CH₂, CHF₂, CH₂Cl, CH₂CH₂CN, CH₂CH₂Cl, cyclopropyl, cyclobutyl, cyclopentyl, cyclohexyl, cyclopropylmethyl, 1-cyclopropylethyl, cyclopentylmethyl, cyclohexylmethyl and phenyl;
L is selected from the group consisting of CH₂, CH₂CH₂, CH(CH₃), CH═CH, CH₂CH₂CH₂, C(CH₃)₂, CH₂CH₂CH₂CH₂, CH₂C(CH₃)₂, CH(CH₃)CH₂CH₂, CH₂CH═CH, C(CH₃)CH═CH₂, CF₂, CHCl and CH₂CHCl;
G is C₃-C₇-cycloalkyl, and in particular cyclopropyl, cyclobutyl, cyclopentyl or cyclohexyl

Specifically preferred are compounds of formula (I-b), wherein
R¹ is methyl or ethyl;
R² is Cl, Br or CN;
R⁵ is CH₃, CH₂CH₃, CH(CH₃)₂ or cyclopropylmethyl,
L is CH₂, CH₂CH₂, CH(CH₃) or C(CH₃)₂;
G is cyclopropyl, cyclobutyl, cyclopentyl or cyclohexyl.

More specifically preferred are compounds of formula (I-b), wherein
R¹ is methyl;
R² is Cl, Br or CN;
R⁵ is CH₃, CH₂CH₃, CH(CH₃)₂ or cyclopropylmethyl,
L is CH₂ or CH(CH₃);
G is cyclopropyl, cyclopentyl or cyclohexyl.

Examples of preferred compounds are the individual compounds compiled in the tables 1 to 42 below, Moreover, the meanings mentioned below for the individual variables in the tables are per se, independently of the combination in which they are mentioned, a particularly preferred embodiment of the substituents in question.

Table 1 Compounds of the formula (I-a) in which R¹ is CH₃, R² is Cl and the combination of R⁵, L and G for a compound corresponds in each case to one row of Table A;

Table 2 Compounds of the formula (I-a) in which R¹ is CH₂CH₃, R² is Cl and the combination of R⁵, L and G for a compound corresponds in each case to one row of Table A;

Table 3 Compounds of the formula (I-a) in which R¹ is CH(CH₃)₂, R² is Cl and the combination of R⁵, L and G for a compound corresponds in each case to one row of Table A;

Table 4 Compounds of the formula (I-a) in which R¹ is cyclopropyl, R² is Cl and the combination of R⁵, L and G for a compound corresponds in each case to one row of Table A;

Table 5 Compounds of the formula (I-a) in which R¹ is cyclobutyl, R² is Cl and the combination of R⁵, L and G for a compound corresponds in each case to one row of Table A;

Table 6 Compounds of the formula (I-a) in which R¹ is cyclopentyl, R² is Cl and the combination of R⁵, L and G for a compound corresponds in each case to one row of Table A;

Table 7 Compounds of the formula (I-a) in which R¹ is cyclohexyl, R² is Cl and the combination of R⁵, L and G for a compound corresponds in each case to one row of Table A;

Table 8 Compounds of the formula (I-a) in which R¹ is CH₃, R² is Br and the combination of R⁵, L and G for a compound corresponds in each case to one row of Table A;

Table 9 Compounds of the formula (I-a) in which R¹ is CH₂CH₃, R² is Br and the combination of R⁵, L and G for a compound corresponds in each case to one row of Table A;

Table 10 Compounds of the formula (I-a) in which R¹ is CH(CH₃)₂, R² is Br and the combination of R⁵, L and G for a compound corresponds in each case to one row of Table A;

Table 11 Compounds of the formula (I-a) in which R¹ is cyclopropyl, R² is Br and the combination of R⁵, L and G for a compound corresponds in each case to one row of Table A;

Table 12 Compounds of the formula (I-a) in which R¹ is cyclobutyl, R² is Br and the combination of R⁵, L and G for a compound corresponds in each case to one row of Table A;

Table 13 Compounds of the formula (I-a) in which R¹ is cyclopentyl, R² is Br and the combination of R⁵, L and G for a compound corresponds in each case to one row of Table A;

Table 14 Compounds of the formula (I-a) in which R¹ is cyclohexyl, R² is Br and the combination of R⁵, L and G for a compound corresponds in each case to one row of Table A;

Table 15 Compounds of the formula (I-a) in which R¹ is CH₃, R² is CN and the combination of R⁵, L and G for a compound corresponds in each case to one row of Table A;

Table 16 Compounds of the formula (I-a) in which R¹ is CH₂CH₃, R² is CN and the combination of R⁵, L and G for a compound corresponds in each case to one row of Table A;

Table 17 Compounds of the formula (I-a) in which R¹ is CH(CH₃)₂, R² is CN and the combination of R⁵, L and G for a compound corresponds in each case to one row of Table A;

Table 18 Compounds of the formula (I-a) in which R¹ is cyclopropyl, R² is CN and the combination of R⁵, L and G for a compound corresponds in each case to one row of Table A;

Table 19 Compounds of the formula (I-a) in which R¹ is cyclobutyl, R² is CN and the combination of R⁵, L and G for a compound corresponds in each case to one row of Table A;

Table 20 Compounds of the formula (I-a) in which R¹ is cyclopentyl, R² is CN and the combination of R⁵, L and G for a compound corresponds in each case to one row of Table A;

Table 21 Compounds of the formula (I-a) in which R¹ is cyclohexyl, R² is CN and the combination of R⁵, L and G for a compound corresponds in each case to one row of Table A.

Table 22 Compounds of the formula (I-b) in which R¹ is CH₃, R² is Cl and the combination of R⁵, L and G for a compound corresponds in each case to one row of Table A;

Table 23 Compounds of the formula (I-b) in which R¹ is CH₂CH₃, R² is Cl and the combination of R⁵, L and G for a compound corresponds in each case to one row of Table A;

Table 24 Compounds of the formula (I-b) in which R¹ is CH(CH₃)₂, R² is Cl and the combination of R⁵, L and G for a compound corresponds in each case to one row of Table A;

Table 25 Compounds of the formula (I-b) in which R¹ is cyclopropyl, R² is Cl and the combination of R⁵, L and G for a compound corresponds in each case to one row of Table A;

Table 26 Compounds of the formula (I-b) in which R¹ is cyclobutyl, R² is Cl and the combination of R⁵, L and G for a compound corresponds in each case to one row of Table A;

Table 27 Compounds of the formula (I-b) in which R¹ is cyclopentyl, R² is Cl and the combination of R⁵, L and G for a compound corresponds in each case to one row of Table A;

Table 28 Compounds of the formula (I-b) in which R¹ is cyclohexyl, R² is Cl and the combination of R⁵, L and G for a compound corresponds in each case to one row of Table A;

Table 29 Compounds of the formula (I-b) in which R¹ is CH₃, R² is Br and the combination of R⁵, L and G for a compound corresponds in each case to one row of Table A;

Table 30 Compounds of the formula (I-b) in which R¹ is CH₂CH₃, R² is Br and the combination of R⁵, L and G for a compound corresponds in each case to one row of Table A;

Table 31 Compounds of the formula (I-b) in which R¹ is CH(CH₃)₂, R² is Br and the combination of R⁵, L and G for a compound corresponds in each case to one row of Table A;

Table 32 Compounds of the formula (I-b) in which R¹ is cyclopropyl, R² is Br and the combination of R⁵, L and G for a compound corresponds in each case to one row of Table A;

Table 33 Compounds of the formula (I-b) in which R¹ is cyclobutyl, R² is Br and the combination of R⁵, L and G for a compound corresponds in each case to one row of Table A;

Table 34 Compounds of the formula (I-b) in which R¹ is cyclopentyl, R² is Br and the combination of R⁵, L and G for a compound corresponds in each case to one row of Table A;

Table 35 Compounds of the formula (I-b) in which R¹ is cyclohexyl, R² is Br and the combination of R⁵, L and G for a compound corresponds in each case to one row of Table A;

Table 36 Compounds of the formula (I-b) in which R¹ is CH₃, R² is CN and the combination of R⁵, L and G for a compound corresponds in each case to one row of Table A;

Table 37 Compounds of the formula (I-b) in which R¹ is CH₂CH₃, R² is CN and the combination of R⁵, L and G for a compound corresponds in each case to one row of Table A;

Table 38 Compounds of the formula (I-b) in which R¹ is CH(CH₃)₂, R² is CN and the combination of R⁵, L and G for a compound corresponds in each case to one row of Table A;

Table 39 Compounds of the formula (I-b) in which R¹ is cyclopropyl, R² is CN and the combination of R⁵, L and G for a compound corresponds in each case to one row of Table A;

Table 40 Compounds of the formula (I-b) in which R¹ is cyclobutyl, R² is CN and the combination of R⁵, L and G for a compound corresponds in each case to one row of Table A;

Table 41 Compounds of the formula (I-b) in which R¹ is cyclopentyl, R² is CN and the combination of R⁵, L and G for a compound corresponds in each case to one row of Table A;

Table 42 Compounds of the formula (I-b) in which R¹ is cyclohexyl, R² is CN and the combination of R⁵, L and G for a compound corresponds in each case to one row of Table A.

TABLE A

| | R⁵ | L-G |
|---|---|---|
| A-1 | CH₃ | CH₂-T1 |
| A-2 | C₂H₅ | CH₂-T1 |
| A-3 | CH═CH₂ | CH₂-T1 |
| A-4 | CH₂CH₂CH₃ | CH₂-T1 |
| A-5 | CH(CH₃)₂ | CH₂-T1 |
| A-6 | CH₂CH₂CH₂CH₃ | CH₂-T1 |
| A-7 | C(CH₃)₃ | CH₂-T1 |
| A-8 | CH₂CH(CH₃)₂ | CH₂-T1 |
| A-9 | CH(CH₃)CH₂CH₃ | CH₂-T1 |
| A-10 | CH₂CH═CH₂ | CH₂-T1 |
| A-11 | CH₂C≡CH | CH₂-T1 |
| A-12 | CH(CH₃)CH═CH₂ | CH₂-T1 |
| A-13 | CHF₂ | CH₂-T1 |
| A-14 | CH₂Cl | CH₂-T1 |
| A-15 | CH₂CH₂CN | CH₂-T1 |
| A-16 | CH₂CH₂Cl | CH₂-T1 |
| A-17 | T1 | CH₂-T1 |
| A-18 | T2 | CH₂-T1 |
| A-19 | T3 | CH₂-T1 |
| A-20 | T4 | CH₂-T1 |
| A-21 | CH₂-T1 | CH₂-T1 |
| A-22 | CH(CH₃)-T1 | CH₂-T1 |
| A-23 | CH₂-T3 | CH₂-T1 |
| A-24 | CH₂-T4 | CH₂-T1 |
| A-25 | C₆H₅ | CH₂-T1 |
| A-26 | CH₃ | CH(CH₃)-T1 |
| A-27 | C₂H₅ | CH(CH₃)-T1 |
| A-28 | CH═CH₂ | CH(CH₃)-T1 |
| A-29 | CH₂CH₂CH₃ | CH(CH₃)-T1 |
| A-30 | CH(CH₃)₂ | CH(CH₃)-T1 |
| A-31 | CH₂CH₂CH₂CH₃ | CH(CH₃)-T1 |
| A-32 | C(CH₃)₃ | CH(CH₃)-T1 |
| A-33 | CH₂CH(CH₃)₂ | CH(CH₃)-T1 |
| A-34 | CH(CH₃)CH₂CH₃ | CH(CH₃)-T1 |
| A-35 | CH₂CH═CH₂ | CH(CH₃)-T1 |
| A-36 | CH₂C≡CH | CH(CH₃)-T1 |
| A-37 | CH(CH₃)CH═CH₂ | CH(CH₃)-T1 |
| A-38 | CHF₂ | CH(CH₃)-T1 |
| A-39 | CH₂Cl | CH(CH₃)-T1 |
| A-40 | CH₂CH₂CN | CH(CH₃)-T1 |
| A-41 | CH₂CH₂Cl | CH(CH₃)-T1 |
| A-42 | T1 | CH(CH₃)-T1 |
| A-43 | T2 | CH(CH₃)-T1 |
| A-44 | T3 | CH(CH₃)-T1 |
| A-45 | T4 | CH(CH₃)-T1 |
| A-46 | CH₂-T1 | CH(CH₃)-T1 |
| A-47 | CH(CH₃)-T1 | CH(CH₃)-T1 |
| A-48 | CH₂-T3 | CH(CH₃)-T1 |
| A-49 | CH₂-T4 | CH(CH₃)-T1 |
| A-50 | C₆H₅ | CH(CH₃)-T1 |
| A-51 | CH₃ | CH═CH-T1 |
| A-52 | C₂H₅ | CH═CH-T1 |
| A-53 | CH═CH₂ | CH═CH-T1 |
| A-54 | CH₂CH₂CH₃ | CH═CH-T1 |
| A-55 | CH(CH₃)₂ | CH═CH-T1 |
| A-56 | CH₂CH₂CH₂CH₃ | CH═CH-T1 |
| A-57 | C(CH₃)₃ | CH═CH-T1 |
| A-58 | CH₂CH(CH₃)₂ | CH═CH-T1 |
| A-59 | CH(CH₃)CH₂CH₃ | CH═CH-T1 |
| A-60 | CH₂CH═CH₂ | CH═CH-T1 |
| A-61 | CH₂C≡CH | CH═CH-T1 |
| A-62 | CH(CH₃)CH═CH₂ | CH═CH-T1 |
| A-63 | CHF₂ | CH═CH-T1 |
| A-64 | CH₂Cl | CH═CH-T1 |
| A-65 | CH₂CH₂CN | CH═CH-T1 |
| A-66 | CH₂CH₂Cl | CH═CH-T1 |
| A-67 | T1 | CH═CH-T1 |
| A-68 | T2 | CH═CH-T1 |
| A-69 | T3 | CH═CH-T1 |
| A-70 | T4 | CH═CH-T1 |
| A-71 | CH₂-T1 | CH═CH-T1 |
| A-72 | CH(CH₃)-T1 | CH═CH-T1 |
| A-73 | CH₂-T3 | CH═CH-T1 |
| A-74 | CH₂-T4 | CH═CH-T1 |
| A-75 | C₆H₅ | CH═CH-T1 |
| A-76 | CH₃ | CH₂CH₂-T1 |
| A-77 | C₂H₅ | CH₂CH₂-T1 |
| A-78 | CH═CH₂ | CH₂CH₂-T1 |
| A-79 | CH₂CH₂CH₃ | CH₂CH₂-T1 |
| A-80 | CH(CH₃)₂ | CH₂CH₂-T1 |
| A-81 | CH₂CH₂CH₂CH₃ | CH₂CH₂-T1 |
| A-82 | C(CH₃)₃ | CH₂CH₂-T1 |
| A-83 | CH₂CH(CH₃)₂ | CH₂CH₂-T1 |
| A-84 | CH(CH₃)CH₂CH₃ | CH₂CH₂-T1 |
| A-85 | CH₂CH═CH₂ | CH₂CH₂-T1 |
| A-86 | CH₂C≡CH | CH₂CH₂-T1 |
| A-87 | CH(CH₃)CH═CH₂ | CH₂CH₂-T1 |
| A-88 | CHF₂ | CH₂CH₂-T1 |
| A-89 | CH₂Cl | CH₂CH₂-T1 |
| A-90 | CH₂CH₂CN | CH₂CH₂-T1 |
| A-91 | CH₂CH₂Cl | CH₂CH₂-T1 |
| A-92 | T1 | CH₂CH₂-T1 |
| A-93 | T2 | CH₂CH₂-T1 |
| A-94 | T3 | CH₂CH₂-T1 |
| A-95 | T4 | CH₂CH₂-T1 |
| A-96 | CH₂-T1 | CH₂CH₂-T1 |
| A-97 | CH(CH₃)-T1 | CH₂CH₂-T1 |
| A-98 | CH₂-T3 | CH₂CH₂-T1 |
| A-99 | CH₂-T4 | CH₂CH₂-T1 |
| A-100 | C₆H₅ | CH₂CH₂-T1 |
| A-101 | CH₃ | CH₂-T2 |
| A-102 | C₂H₅ | CH₂-T2 |
| A-103 | CH═CH₂ | CH₂-T2 |
| A-104 | CH₂CH₂CH₃ | CH₂-T2 |
| A-105 | CH(CH₃)₂ | CH₂-T2 |
| A-106 | CH₂CH₂CH₂CH₃ | CH₂-T2 |
| A-107 | C(CH₃)₃ | CH₂-T2 |
| A-108 | CH₂CH(CH₃)₂ | CH₂-T2 |
| A-109 | CH(CH₃)CH₂CH₃ | CH₂-T2 |
| A-110 | CH₂CH═CH₂ | CH₂-T2 |
| A-111 | CH₂C≡CH | CH₂-T2 |
| A-112 | CH(CH₃)CH═CH₂ | CH₂-T2 |
| A-113 | CHF₂ | CH₂-T2 |
| A-114 | CH₂Cl | CH₂-T2 |
| A-115 | CH₂CH₂CN | CH₂-T2 |
| A-116 | CH₂CH₂Cl | CH₂-T2 |
| A-117 | T1 | CH₂-T2 |
| A-118 | T2 | CH₂-T2 |
| A-119 | T3 | CH₂-T2 |
| A-120 | T4 | CH₂-T2 |
| A-121 | CH₂-T1 | CH₂-T2 |
| A-122 | CH(CH₃)-T1 | CH₂-T2 |
| A-123 | CH₂-T3 | CH₂-T2 |
| A-124 | CH₂-T4 | CH₂-T2 |
| A-125 | C₆H₅ | CH₂-T2 |
| A-126 | CH₃ | CH(CH₃)-T2 |
| A-127 | C₂H₅ | CH(CH₃)-T2 |
| A-128 | CH═CH₂ | CH(CH₃)-T2 |
| A-129 | CH₂CH₂CH₃ | CH(CH₃)-T2 |
| A-130 | CH(CH₃)₂ | CH(CH₃)-T2 |
| A-131 | CH₂CH₂CH₂CH₃ | CH(CH₃)-T2 |
| A-132 | C(CH₃)₃ | CH(CH₃)-T2 |
| A-133 | CH₂CH(CH₃)₂ | CH(CH₃)-T2 |
| A-134 | CH(CH₃)CH₂CH₃ | CH(CH₃)-T2 |
| A-135 | CH₂CH═CH₂ | CH(CH₃)-T2 |
| A-136 | CH₂C≡CH | CH(CH₃)-T2 |
| A-137 | CH(CH₃)CH═CH₂ | CH(CH₃)-T2 |
| A-138 | CHF₂ | CH(CH₃)-T2 |
| A-139 | CH₂Cl | CH(CH₃)-T2 |
| A-140 | CH₂CH₂CN | CH(CH₃)-T2 |
| A-141 | CH₂CH₂Cl | CH(CH₃)-T2 |
| A-142 | T1 | CH(CH₃)-T2 |
| A-143 | T2 | CH(CH₃)-T2 |
| A-144 | T3 | CH(CH₃)-T2 |
| A-145 | T4 | CH(CH₃)-T2 |
| A-146 | CH₂-T1 | CH(CH₃)-T2 |
| A-147 | CH(CH₃)-T1 | CH(CH₃)-T2 |
| A-148 | CH₂-T3 | CH(CH₃)-T2 |
| A-149 | CH₂-T4 | CH(CH₃)-T2 |
| A-150 | C₆H₅ | CH(CH₃)-T2 |
| A-151 | CH₃ | CH═CH-T2 |
| A-152 | C₂H₅ | CH═CH-T2 |
| A-153 | CH═CH₂ | CH═CH-T2 |
| A-154 | CH₂CH₂CH₃ | CH═CH-T2 |
| A-155 | CH(CH₃)₂ | CH═CH-T2 |
| A-156 | CH₂CH₂CH₂CH₃ | CH═CH-T2 |
| A-157 | C(CH₃)₃ | CH═CH-T2 |
| A-158 | CH₂CH(CH₃)₂ | CH═CH-T2 |
| A-159 | CH(CH₃)CH₂CH₃ | CH═CH-T2 |
| A-160 | CH₂CH═CH₂ | CH═CH-T2 |
| A-161 | CH₂C≡CH | CH═CH-T2 |
| A-162 | CH(CH₃)CH═CH₂ | CH═CH-T2 |
| A-163 | CHF₂ | CH═CH-T2 |
| A-164 | CH₂Cl | CH═CH-T2 |
| A-165 | CH₂CH₂CN | CH═CH-T2 |
| A-166 | CH₂CH₂Cl | CH═CH-T2 |
| A-167 | T1 | CH═CH-T2 |
| A-168 | T2 | CH═CH-T2 |
| A-169 | T3 | CH═CH-T2 |
| A-170 | T4 | CH═CH-T2 |
| A-171 | CH₂-T1 | CH═CH-T2 |
| A-172 | CH(CH₃)-T1 | CH═CH-T2 |
| A-173 | CH₂-T3 | CH═CH-T2 |
| A-174 | CH₂-T4 | CH═CH-T2 |
| A-175 | C₆H₅ | CH═CH-T2 |
| A-176 | CH₃ | CH₂CH₂-T2 |
| A-177 | C₂H₅ | CH₂CH₂-T2 |
| A-178 | CH═CH₂ | CH₂CH₂-T2 |
| A-179 | CH₂CH₂CH₃ | CH₂CH₂-T2 |
| A-180 | CH(CH₃)₂ | CH₂CH₂-T2 |
| A-181 | CH₂CH₂CH₂CH₃ | CH₂CH₂-T2 |
| A-182 | C(CH₃)₃ | CH₂CH₂-T2 |
| A-183 | CH₂CH(CH₃)₂ | CH₂CH₂-T2 |
| A-184 | CH(CH₃)CH₂CH₃ | CH₂CH₂-T2 |
| A-185 | CH₂CH═CH₂ | CH₂CH₂-T2 |
| A-186 | CH₂C≡CH | CH₂CH₂-T2 |
| A-187 | CH(CH₃)CH═CH₂ | CH₂CH₂-T2 |
| A-188 | CHF₂ | CH₂CH₂-T2 |
| A-189 | CH₂Cl | CH₂CH₂-T2 |
| A-190 | CH₂CH₂CN | CH₂CH₂-T2 |
| A-191 | CH₂CH₂Cl | CH₂CH₂-T2 |
| A-192 | T1 | CH₂CH₂-T2 |
| A-193 | T2 | CH₂CH₂-T2 |
| A-194 | T3 | CH₂CH₂-T2 |
| A-195 | T4 | CH₂CH₂-T2 |
| A-196 | CH₂-T1 | CH₂CH₂-T2 |
| A-197 | CH(CH₃)-T1 | CH₂CH₂-T2 |
| A-198 | CH₂-T3 | CH₂CH₂-T2 |
| A-199 | CH₂-T4 | CH₂CH₂-T2 |
| A-200 | C₆H₅ | CH₂CH₂-T2 |
| A-201 | CH₃ | CH₂-T3 |
| A-202 | C₂H₅ | CH₂-T3 |
| A-203 | CH═CH₂ | CH₂-T3 |
| A-204 | CH₂CH₂CH₃ | CH₂-T3 |
| A-205 | CH(CH₃)₂ | CH₂-T3 |
| A-206 | CH₂CH₂CH₂CH₃ | CH₂-T3 |
| A-207 | C(CH₃)₃ | CH₂-T3 |
| A-208 | CH₂CH(CH₃)₂ | CH₂-T3 |
| A-209 | CH(CH₃)CH₂CH₃ | CH₂-T3 |
| A-210 | CH₂CH═CH₂ | CH₂-T3 |
| A-211 | CH₂C≡CH | CH₂-T3 |
| A-212 | CH(CH₃)CH═CH₂ | CH₂-T3 |
| A-213 | CHF₂ | CH₂-T3 |
| A-214 | CH₂Cl | CH₂-T3 |
| A-215 | CH₂CH₂CN | CH₂-T3 |
| A-216 | CH₂CH₂Cl | CH₂-T3 |
| A-217 | T1 | CH₂-T3 |
| A-218 | T2 | CH₂-T3 |
| A-219 | T3 | CH₂-T3 |
| A-220 | T4 | CH₂-T3 |
| A-221 | CH₂-T1 | CH₂-T3 |
| A-222 | CH(CH₃)-T1 | CH₂-T3 |
| A-223 | CH₂-T3 | CH₂-T3 |
| A-224 | CH₂-T4 | CH₂-T3 |
| A-225 | C₆H₅ | CH₂-T3 |
| A-226 | CH₃ | CH(CH₃)-T3 |
| A-227 | C₂H₅ | CH(CH₃)-T3 |
| A-228 | CH═CH₂ | CH(CH₃)-T3 |
| A-229 | CH₂CH₂CH₃ | CH(CH₃)-T3 |
| A-230 | CH(CH₃)₂ | CH(CH₃)-T3 |
| A-231 | CH₂CH₂CH₂CH₃ | CH(CH₃)-T3 |
| A-232 | C(CH₃)₃ | CH(CH₃)-T3 |
| A-233 | CH₂CH(CH₃)₂ | CH(CH₃)-T3 |
| A-234 | CH(CH₃)CH₂CH₃ | CH(CH₃)-T3 |
| A-235 | CH₂CH═CH₂ | CH(CH₃)-T3 |
| A-236 | CH₂C≡CH | CH(CH₃)-T3 |
| A-237 | CH(CH₃)CH═CH₂ | CH(CH₃)-T3 |
| A-238 | CHF₂ | CH(CH₃)-T3 |
| A-239 | CH₂Cl | CH(CH₃)-T3 |
| A-240 | CH₂CH₂CN | CH(CH₃)-T3 |
| A-241 | CH₂CH₂Cl | CH(CH₃)-T3 |
| A-242 | T1 | CH(CH₃)-T3 |
| A-243 | T2 | CH(CH₃)-T3 |
| A-244 | T3 | CH(CH₃)-T3 |
| A-245 | T4 | CH(CH₃)-T3 |
| A-246 | CH₂-T1 | CH(CH₃)-T3 |
| A-247 | CH(CH₃)-T1 | CH(CH₃)-T3 |
| A-248 | CH₂-T3 | CH(CH₃)-T3 |
| A-249 | CH₂-T4 | CH(CH₃)-T3 |
| A-250 | C₆H₅ | CH(CH₃)-T3 |
| A-251 | CH₃ | CH═CH-T3 |
| A-252 | C₂H₅ | CH═CH-T3 |
| A-253 | CH═CH₂ | CH═CH-T3 |
| A-254 | CH₂CH₂CH₃ | CH═CH-T3 |
| A-255 | CH(CH₃)₂ | CH═CH-T3 |
| A-256 | CH₂CH₂CH₂CH₃ | CH═CH-T3 |
| A-257 | C(CH₃)₃ | CH═CH-T3 |
| A-258 | CH₂CH(CH₃)₂ | CH═CH-T3 |
| A-259 | CH(CH₃)CH₂CH₃ | CH═CH-T3 |
| A-260 | CH₂CH═CH₂ | CH═CH-T3 |
| A-261 | CH₂C≡CH | CH═CH-T3 |
| A-262 | CH(CH₃)CH═CH₂ | CH═CH-T3 |
| A-263 | CHF₂ | CH═CH-T3 |
| A-264 | CH₂Cl | CH═CH-T3 |
| A-265 | CH₂CH₂CN | CH═CH-T3 |
| A-266 | CH₂CH₂Cl | CH═CH-T3 |
| A-267 | T1 | CH═CH-T3 |
| A-268 | T2 | CH═CH-T3 |
| A-269 | T3 | CH═CH-T3 |
| A-270 | T4 | CH═CH-T3 |
| A-271 | CH₂-T1 | CH═CH-T3 |
| A-272 | CH(CH₃)-T1 | CH═CH-T3 |
| A-273 | CH₂-T3 | CH═CH-T3 |
| A-274 | CH₂-T4 | CH═CH-T3 |
| A-275 | C₆H₅ | CH═CH-T3 |
| A-276 | CH₃ | CH₂CH₂-T3 |
| A-277 | C₂H₅ | CH₂CH₂-T3 |
| A-278 | CH═CH₂ | CH₂CH₂-T3 |
| A-279 | CH₂CH₂CH₃ | CH₂CH₂-T3 |
| A-280 | CH(CH₃)₂ | CH₂CH₂-T3 |
| A-281 | CH₂CH₂CH₂CH₃ | CH₂CH₂-T3 |
| A-282 | C(CH₃)₃ | CH₂CH₂-T3 |
| A-283 | CH₂CH(CH₃)₂ | CH₂CH₂-T3 |
| A-284 | CH(CH₃)CH₂CH₃ | CH₂CH₂-T3 |
| A-285 | CH₂CH═CH₂ | CH₂CH₂-T3 |
| A-286 | CH₂C≡CH | CH₂CH₂-T3 |
| A-287 | CH(CH₃)CH═CH₂ | CH₂CH₂-T3 |
| A-288 | CHF₂ | CH₂CH₂-T3 |
| A-289 | CH₂Cl | CH₂CH₂-T3 |
| A-290 | CH₂CH₂CN | CH₂CH₂-T3 |
| A-291 | CH₂CH₂Cl | CH₂CH₂-T3 |
| A-292 | T1 | CH₂CH₂-T3 |
| A-293 | T2 | CH₂CH₂-T3 |
| A-294 | T3 | CH₂CH₂-T3 |
| A-295 | T4 | CH₂CH₂-T3 |
| A-296 | CH₂-T1 | CH₂CH₂-T3 |
| A-297 | CH(CH₃)-T1 | CH₂CH₂-T3 |
| A-298 | CH₂-T3 | CH₂CH₂-T3 |
| A-299 | CH₂-T4 | CH₂CH₂-T3 |
| A-300 | C₆H₅ | CH₂CH₂-T3 |
| A-301 | CH₃ | CH₂-T4 |
| A-302 | C₂H₅ | CH₂-T4 |
| A-303 | CH═CH₂ | CH₂-T4 |
| A-304 | CH₂CH₂CH₃ | CH₂-T4 |
| A-305 | CH(CH₃)₂ | CH₂-T4 |
| A-306 | CH₂CH₂CH₂CH₃ | CH₂-T4 |
| A-307 | C(CH₃)₃ | CH₂-T4 |
| A-308 | CH₂CH(CH₃)₂ | CH₂-T4 |
| A-309 | CH(CH₃)CH₂CH₃ | CH₂-T4 |
| A-310 | CH₂CH═CH₂ | CH₂-T4 |
| A-311 | CH₂C≡CH | CH₂-T4 |
| A-312 | CH(CH₃)CH═CH₂ | CH₂-T4 |
| A-313 | CHF₂ | CH₂-T4 |
| A-314 | CH₂Cl | CH₂-T4 |
| A-315 | CH₂CH₂CN | CH₂-T4 |
| A-316 | CH₂CH₂Cl | CH₂-T4 |
| A-317 | T1 | CH₂-T4 |
| A-318 | T2 | CH₂-T4 |
| A-319 | T3 | CH₂-T4 |
| A-320 | T4 | CH₂-T4 |
| A-321 | CH₂-T1 | CH₂-T4 |
| A-322 | CH(CH₃)-T1 | CH₂-T4 |
| A-323 | CH₂-T3 | CH₂-T4 |
| A-324 | CH₂-T4 | CH₂-T4 |
| A-325 | C₆H₅ | CH₂-T4 |
| A-326 | CH₃ | CH(CH₃)-T4 |
| A-327 | C₂H₅ | CH(CH₃)-T4 |
| A-328 | CH═CH₂ | CH(CH₃)-T4 |
| A-329 | CH₂CH₂CH₃ | CH(CH₃)-T4 |
| A-330 | CH(CH₃)₂ | CH(CH₃)-T4 |
| A-331 | CH₂CH₂CH₂CH₃ | CH(CH₃)-T4 |
| A-332 | C(CH₃)₃ | CH(CH₃)-T4 |
| A-333 | CH₂CH(CH₃)₂ | CH(CH₃)-T4 |
| A-334 | CH(CH₃)CH₂CH₃ | CH(CH₃)-T4 |
| A-335 | CH₂CH═CH₂ | CH(CH₃)-T4 |
| A-336 | CH₂C≡CH | CH(CH₃)-T4 |
| A-337 | CH(CH₃)CH═CH₂ | CH(CH₃)-T4 |
| A-338 | CHF₂ | CH(CH₃)-T4 |
| A-339 | CH₂Cl | CH(CH₃)-T4 |
| A-340 | CH₂CH₂CN | CH(CH₃)-T4 |
| A-341 | CH₂CH₂Cl | CH(CH₃)-T4 |
| A-342 | T1 | CH(CH₃)-T4 |
| A-343 | T2 | CH(CH₃)-T4 |
| A-344 | T3 | CH(CH₃)-T4 |
| A-345 | T4 | CH(CH₃)-T4 |
| A-346 | CH₂-T1 | CH(CH₃)-T4 |
| A-347 | CH(CH₃)-T1 | CH(CH₃)-T4 |
| A-348 | CH₂-T3 | CH(CH₃)-T4 |
| A-349 | CH₂-T4 | CH(CH₃)-T4 |
| A-350 | C₆H₅ | CH(CH₃)-T4 |
| A-351 | CH₃ | CH═CH-T4 |
| A-352 | C₂H₅ | CH═CH-T4 |
| A-353 | CH═CH₂ | CH═CH-T4 |
| A-354 | CH₂CH₂CH₃ | CH═CH-T4 |
| A-355 | CH(CH₃)₂ | CH═CH-T4 |
| A-356 | CH₂CH₂CH₂CH₃ | CH═CH-T4 |
| A-357 | C(CH₃)₃ | CH═CH-T4 |
| A-358 | CH₂CH(CH₃)₂ | CH═CH-T4 |
| A-359 | CH(CH₃)CH₂CH₃ | CH═CH-T4 |
| A-360 | CH₂CH═CH₂ | CH═CH-T4 |
| A-361 | CH₂C≡CH | CH═CH-T4 |
| A-362 | CH(CH₃)CH═CH₂ | CH═CH-T4 |
| A-363 | CHF₂ | CH═CH-T4 |
| A-364 | CH₂Cl | CH═CH-T4 |
| A-365 | CH₂CH₂CN | CH═CH-T4 |
| A-366 | CH₂CH₂Cl | CH═CH-T4 |
| A-367 | T1 | CH═CH-T4 |
| A-368 | T2 | CH═CH-T4 |
| A-369 | T3 | CH═CH-T4 |
| A-370 | T4 | CH═CH-T4 |
| A-371 | CH₂-T1 | CH═CH-T4 |
| A-372 | CH(CH₃)-T1 | CH═CH-T4 |
| A-373 | CH₂-T3 | CH═CH-T4 |
| A-374 | CH₂-T4 | CH═CH-T4 |
| A-375 | C₆H₅ | CH═CH-T4 |
| A-376 | CH₃ | CH₂CH₂-T4 |
| A-377 | C₂H₅ | CH₂CH₂-T4 |
| A-378 | CH═CH₂ | CH₂CH₂-T4 |
| A-379 | CH₂CH₂CH₃ | CH₂CH₂-T4 |
| A-380 | CH(CH₃)₂ | CH₂CH₂-T4 |
| A-381 | CH₂CH₂CH₂CH₃ | CH₂CH₂-T4 |
| A-382 | C(CH₃)₃ | CH₂CH₂-T4 |
| A-383 | CH₂CH(CH₃)₂ | CH₂CH₂-T4 |
| A-384 | CH(CH₃)CH₂CH₃ | CH₂CH₂-T4 |
| A-385 | CH₂CH═CH₂ | CH₂CH₂-T4 |
| A-386 | CH₂C≡CH | CH₂CH₂-T4 |
| A-387 | CH(CH₃)CH═CH₂ | CH₂CH₂-T4 |
| A-388 | CHF₂ | CH₂CH₂-T4 |
| A-389 | CH₂Cl | CH₂CH₂-T4 |
| A-390 | CH₂CH₂CN | CH₂CH₂-T4 |
| A-391 | CH₂CH₂Cl | CH₂CH₂-T4 |
| A-392 | T1 | CH₂CH₂-T4 |
| A-393 | T2 | CH₂CH₂-T4 |
| A-394 | T3 | CH₂CH₂-T4 |
| A-395 | T4 | CH₂CH₂-T4 |
| A-396 | CH₂-T1 | CH₂CH₂-T4 |
| A-397 | CH(CH₃)-T1 | CH₂CH₂-T4 |
| A-398 | CH₂-T3 | CH₂CH₂-T4 |
| A-399 | CH₂-T4 | CH₂CH₂-T4 |
| A-400 | C₆H₅ | CH₂CH₂-T4 |

T1: cyclopropyl; T2: cyclobutyl; T3: cyclopentyl; T4: cyclohexyl.

The compounds of the formula (I) can be prepared by the standard methods of organic chemistry, e.g. by the methods described hereinafter in schemes 1 to 6 and in the synthesis descriptions of the working examples. The substituents, variables and indices in schemes 1 to 6 are as defined above for formula (I), if not otherwise specified.

The compounds of formula (I) can be prepared as shown in the Scheme 1 below.

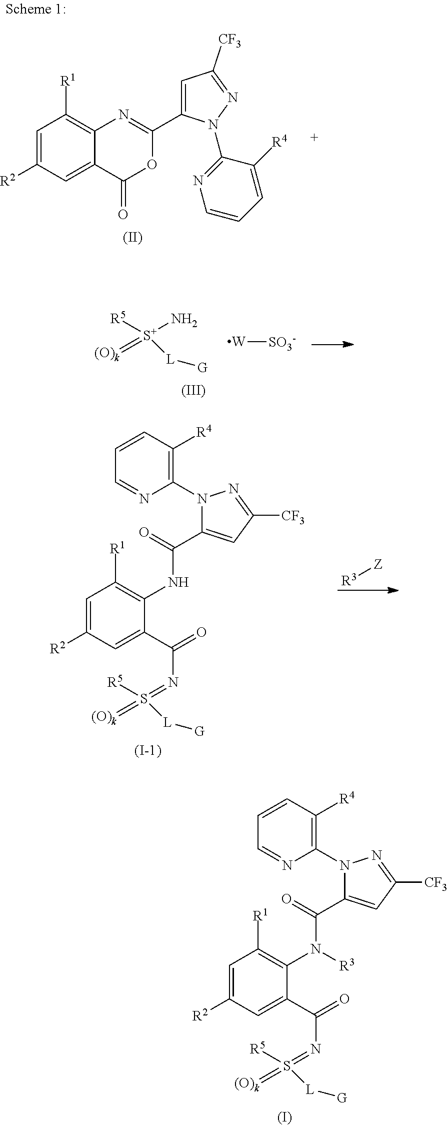

Compounds of formula (II) are reacted with compounds of formula (III), in which W can be any group which does not disturb the reaction, such as OH, NH₂, optionally substituted alkyl, optionally substituted aryl or optionally substituted hetaryl, but which is preferably an aromatic group, such as phenyl, optionally substituted with one or more radicals such as defined as Rf, for example 2,4,6-trimethylphenyl, to give compounds of formula (I-1). The reaction is suitably carried out in a polar or apolar aprotic solvent, such as N,N-dimethylformamide, tetrahydrofuran, dioxane, acetonitrile, dimethylsulfoxide, pyridine, dichloromethane, benzene, toluene, the xylenes or chlorobenzene or mixtures of such solvents, in a temperature range of from 0° C. and 100° C., preferably of from 20° C. and 90° C. The reaction is suitably carried out in the presence of a base. Suitable bases include but are not limited to oxo bases and amine bases. Suitable oxo bases include but are not limited to hydroxides, in particular alkalimetal hydroxides such as lithium, sodium or potassium hydroxide, carbonates, in particular alkalimetal carbonates, such as lithium, sodium or potassium carbonates, hydrogen carbonates, in particular alkalimetal hydrogen carbonates, such as lithium, sodium or potassium hydrogen carbonates, phosphates or hydrogenphosphates, in particular alkalimetal phosphates or hydrogenphosphates, such as lithium, sodium or potassium phosphate, or lithium, sodium or potassium hydrogen phosphate, alkoxides, in particular alkalimetal alkoxides such as sodium or potassium methoxide, sodium or potassium ethoxide or sodium or potassium tert-butanolate, carboxylates, in particular alkalimetal carboxylates, such as lithium, sodium or potassium formiate, lithium, sodium or potassium acetate or lithium, sodium or potassium propionate. Suitable amine bases include but are not limited to ammonia and organic amines, in particular aliphatic or cycloaliphatic amines, e.g. di-C₁-C₄-alkylamines, tri-C₁-C₄-alkylamines, C₃-C₆-cycloalkylamines, C₃-C₆-cycloalkyl-di-C₁-C₄-alkylamines or cyclic amines such as dimethylamine, diethylamine, diisopropylamine, cyclohexylamine, dimethylcyclohexylamine, trimethylamine, diethylamine or triethylamine, piperidine and N-methylpiperidine. Preferred bases are oxo bases, in particular alkalimetal alkoxides, which are also termed alkalimetal alkanolates, especially sodium and potassium alkanolates such as sodium methoxides, potassium methoxide, sodium ethoxide, potassium ethoxide, sodium tert-butanolate or potassium tert-butanolate. Mixtures of oxobases and amine bases may also be used. Compound of formula (III) is typically employed in an amount of from 0.9 to 5 mol, preferably from 0.9 to 3 mol, more preferably from 0.9 to 1.5 mol and in particular from 0.95 to 1.2 mol per mol of the compound of formula (II) used.

For converting compounds of formula (I-1) into compounds (I) in which R³ is not H, compounds of formula (I-1) can be reacted with compounds of formula R³—Z, wherein R³ is not H and Z is a leaving group, such as for example a bromine, chlorine or iodine atom or a tosylate, mesylate or triflate, to give compounds of formula (I). The reaction is suitably carried out in the presence of a base such as sodium hydride or potassium hydride, suitably in a polar aprotic solvent such as N,N-dimethylformamide, tetrahydrofuran, dioxane, acetonitrile, dimethylsulfoxide or pyridine, or mixtures of these solvents, in a temperature range of from 0° C. and 100 C. In case k is 0 in compounds of formulae (I-1) or (I), a subsequent oxidation reaction in analogy to methods described for example by Dillard et al, Journal of Medicinal Chemistry (1980), 23, 717-722, may be performed to yield compounds of the aforementioned formulae (I-1) or (I), in which k is 1. Other preparation methods for compounds of formula (I) may also be adapted from analogous reactions, as for example described in WO 2007/006670.

Compounds of formula (III) can be obtained as shown in Scheme 2 below.

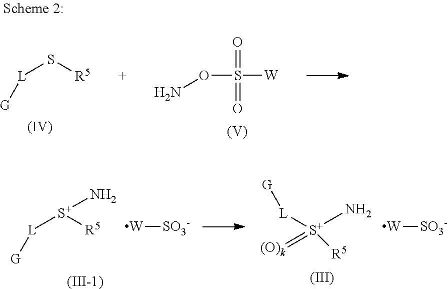

Reaction of a sulfonyl hydroxylamine of formula (V), in which W is as defined for scheme 1 and is preferably an aromatic group such as phenyl, optionally substituted with one or more radicals, such as defined as Rf, with a sulfide of formula (IV) yields compounds of formula (III-1), corresponding to compounds of formula III in which k is 0, which is described in more detail e.g. by Fujii et al., Heteroatom Chemistry (2004), 15(3), 246-250 or by Young et al, Journal of Organic Chemistry, 1987, (52), 2695-2699. The reaction may also be carried out in analogy to reactions known from literature, in which R⁵ and LG have other meanings than in the present invention. In analogy to the described methods an amination reaction of the sulfide of formula (IV) may also be accomplished by applying reagents such as sulfoperamidic acid (W═OH). Compounds of formula (III), in which k is 1, may be obtained from compounds of formula (III-1) by oxidation with an appropriate oxidant, in analogy to described methods as described by, for example, Dillard et al, Journal of Medicinal Chemistry (1980), 23, 717-722. Further preparation methods may also be found in WO 2007/006670 and the references cited therein.

Alternatively, compounds of formula (I), in which k is 0, can also be prepared as shown in scheme 3. Reaction of a compound of formula (VI) with an activated sulfoxide of formula (VII) yields a compound of formula (I), in which k is 0, in analogy to those reactions known from literature, in which the substituents have other meanings than in the present invention, as for example described by Sharma et al, Journal of Organic Chemistry (1975), 40, 2758-2764. Compounds of formula (VI) can be prepared in analogy to the methods described in WO 2009/085816.

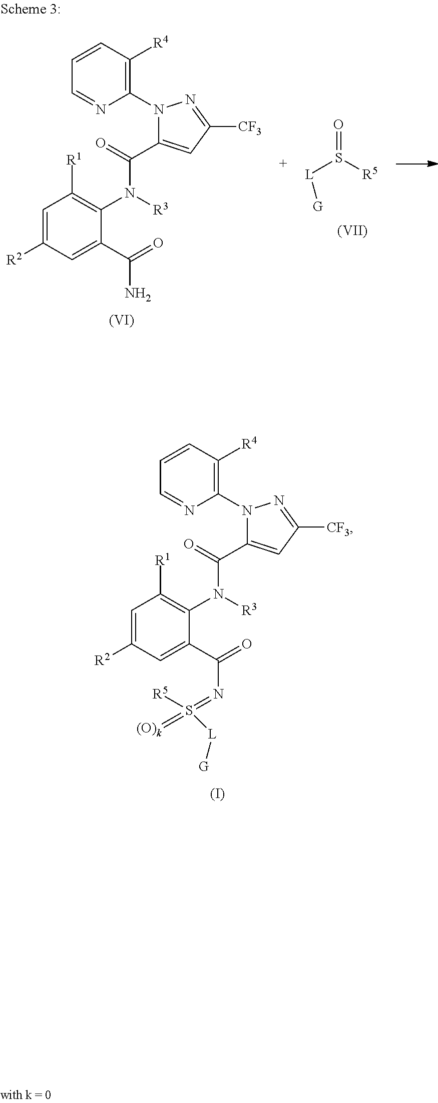

Alternatively, compounds of formula (I) can also be prepared as shown in scheme 4. Reaction of a compound of formula (VI) with a sulfide of formula (IV) yields a compound of formula (I), in which k is 0, in analogy to methods known in the literature, e.g. Ried et al, Chemische Berichte (1984), 117, 2779-2784. The compound of formula (I), in which k is 0, can be further oxidized by known methods to a compound of formula (I), in which k is 1.

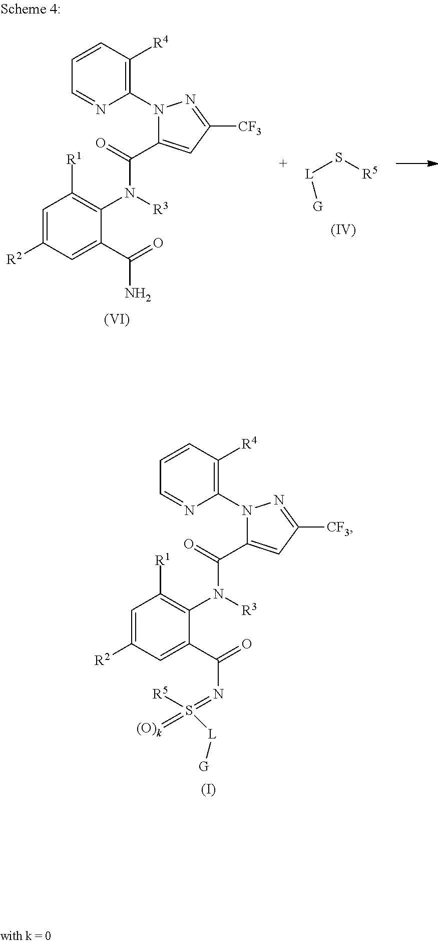

Alternatively, compounds of formula (I) can also be prepared as shown in scheme 5. Reaction of a compound of formula (VII) with a carboxylic acid derivative (VIII) yields compound (I). Z is a leaving group, such as halogen, in particular Cl, an anhydride residue or an active ester residue. Especially in case of Z being halogen the reaction is suitably carried out in the presence of a base. Suitable bases are for example carbonates, such as lithium, sodium or potassium carbonates, amines, such as trimethylamine or triethylamine, and basic N-heterocycles, such as pyridine, 2,6-dimethylpyridine or 2,4,6-trimethylpyridine. Suitable solvents are in particular aprotic solvents such as pentane, hexane, heptane, octane, cyclohexane, dichloromethane, chloroform, 1,2-dichlorethane, benzene, chlorobenzene, toluene, the xylenes, dichlorobenzene, trimethylbenzene, pyridine, 2,6-dimethylpyridine, 2,4,6-trimethylpyridine, acetonitrile, diethyl ether, tetrahydrofuran, 2-methyl tetrahydrofuran, methyl tert-butylether, 1,4-dioxane, N,N-dimethyl formamide, N-methylpyrrolidinone or mixtures thereof.

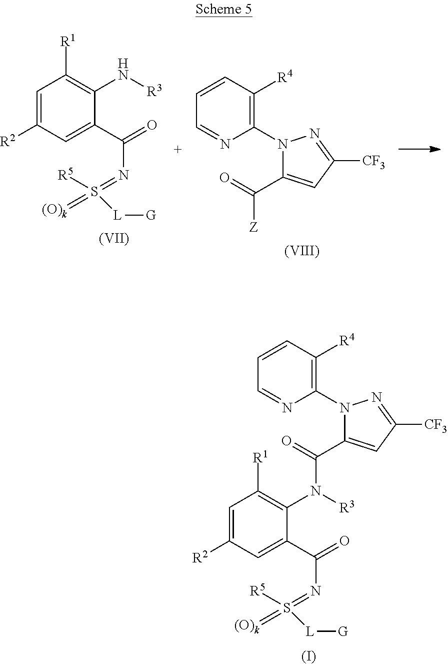

As shown in scheme 6 below the compound of formula (VII) can be obtained by reacting the benzoxazinone (IX) with the sulfinium compound of formula (X) or with the sulfinimin salt (III′) which may be an aforementioned compound of formula (III). A⁻ is the equivalent of an anion, preferably of an anion having a pK_{B} of at least 10, as determined under standard conditions (298 K, 1.103 bar) in water. Anion equivalent means the amount of anion required to achieve electroneutrality. For example, if the anion carries one negative charge the equivalent is 1, while if the anione carries two negative charges the equivalent is ½. Suitable anions include inorganic ions such as SO₄²⁻, HSO₄⁻, Cl⁻, ClO₄⁻, BF₄⁻, PF₆⁻, HPO₄⁻, and organic anions such as methylsulfonate, trifluoromethylsulfonate, trifluoroacetate, phenylsulfonate, toluenesulfonate, mesitylene sulfonate and the like. The reaction is suitably carried out in the presence of a base. Suitable bases include hydroxides, such as lithium, sodium or potassium hydroxide, carbonates, such as lithium, sodium or potassium carbonates, hydrogen carbonates, such as lithium, sodium or potassium hydrogen carbonates, phosphates, such as lithium, sodium or potassium phosphate, hydrogen phosphate, such as lithium, sodium or potassium hydrogen phosphate, alkoxides, such as sodium or potassium methoxide, sodium or potassium ethoxide or sodium or potassium tert-butanolate, carboxylates, such as lithium, sodium or potassium formiate, lithium, sodium or potassium acetate or lithium, sodium or potassium propionate, ammonia and amines, such as dimethylamine, trimethylamine, diethylamine or triethylamine. Suitable solvents can be protic or aprotic. Examples for aprotic solvents are aliphatic hydrocarbons, such as alkanes, e.g. pentane, hexane or heptane, cycloaliphatic hydrocarbons, such as cycloalkanes, e.g. cyclopentane or cyclohexane, halogenated alkanes, such as methylene chloride, chloroform or 1,2-dichlorethane, aromatic hydrocarbons, such as benzene, toluene, the xylenes or chlorobenzene, open-chained ethers, such as diethylether, methyl-tert-butyl ether or methyl-isobutyl ether, cyclic ethers, such as tetrahydrofuran, 1,4-dioxane or 2-methyl tetrahydrofuran, or esters, such as ethyl acetate or ethyl propionate. Furthermore, pyridine, 2,6-dimethylpyridine, 2,4,6-trimethylpyridine, N,N-dimethyl formamide, N-methylpyrrolidinone or mixtures of solvents mentioned above or below are suitable. Examples for polar protic solvents are C₁-C₄-alcohols such as methanol, ethanol, propanol and isopropanol, glycols, such as ethylene glycol and diethylene glycol, and mixtures thereof.

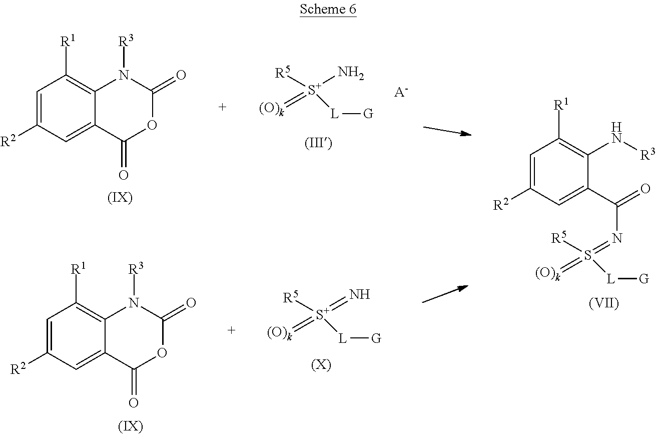

The compound of formula (III′) can be prepared by reacting a sulfide or sulfoxide S(O)ₖR⁵LG with an amination agent, such as aminoxysulfonic acid NH₂OSO₃H. The preparation via the sulfide can be carried out in accordance to scheme 2.

As a rule, the compounds of formula (I) including their stereoisomers, salts, tautomers and N-oxides, and their precursors in the synthesis process [especially (I-1), (II), (III), (III-1), (IV), (V), (VI), (VII)], can be prepared by the methods described above. If individual compounds can not be prepared via the above-described routes, they can be prepared by derivatization of other compounds (I) or the respective precursor or by customary modifications of the synthesis routes described. For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

Due to their excellent activity, the compounds of the present invention may be used for controlling invertebrate pests.

Accordingly, the present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of the present invention or a composition as defined above.

Preferably, the method of the invention serves for protecting plant propagation material (such as seed) and the plant which grows therefrom from invertebrate pest attack or infestation and comprises treating the plant propagation material (such as seed) with a pesticidally effective amount of a compound of the present invention as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

In the sense of the present invention, “invertebrate pests” are preferably selected from arthropods and nematodes, more preferably from harmful insects, arachnids and nematodes, and even more preferably from insects, acarids and nematodes. In the sense of the present invention, “invertebrate pests” are most preferably insects.

The invention further provides an agricultural composition for combating invertebrate pests, which comprises such an amount of at least one compound according to the invention and at least one inert liquid and/or solid agronomically acceptable carrier that has a pesticidal action and, if desired, at least one surfactant.

Such a composition may comprise a single active compound of the present invention or a mixture of several active compounds of the present invention. The composition according to the present invention may comprise an individual isomer or mixtures of isomers or a salt as well as individual tautomers or mixtures of tautomers.

The compounds of the present invention, including their salts, stereoisomers and tautomers, are in particular suitable for efficiently controlling arthropodal pests such as arachnids, myriapedes and insects as well as nematodes. They are especially suitable for efficiently combating or controlling the following pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Pieris rapae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria;
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;
thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;
bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus;
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;
arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;
fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Scutigera coleoptrata,
millipedes (Diplopoda), e.g. Narceus spp.,
earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
Collembola (springtails), e.g. Onychiurus ssp.

The compounds of the present invention, including their salts, stereoisomers and tautomers, are also suitable for controlling nematodes:plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the present invention, including their salts, stereoisomers and tautomers, are particularly useful for controlling insects, preferably sucking or piercing and chewing and biting insects such as insects from the genera Lepidoptera, Coleoptera and Hemiptera, in particular Lepidoptera, Coleoptera and true bugs.

The compounds of the present invention, including their salts, stereoisomers and tautomers, are moreover useful for controlling insects of the orders Thysanoptera, Diptera (especially flies, mosquitos), Hymenoptera (especially ants) and Isoptera (especially termites.

The compounds of the present invention, including their salts, stereoisomers and tautomers, are particularly useful for controlling insects of the orders Lepidoptera and Coleoptera.

The compounds of the present invention can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the present invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineers Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Solvents/carriers, which are suitable, are e.g.:

solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones [N-methyl-pyrrolidone (NMP), N-octylpyrrolidone (NOP)], acetates (glycol diacetate), alkyl lactates, lactones such as gamma-butyrolactone, glycols, fatty acid dimethylamides, fatty acids and fatty acid esters, triglycerides, oils of vegetable or animal origin and modified oils such as alkylated plant oils. In principle, solvent mixtures may also be used;

carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters,

Also anti-freezing agents, such as glycerin, ethylene glycol or propylene glycol, and bactericides, such as bronopol and isothiazolinone derivatives such as alkylisothiazolinones and benzisothiazolinones, can be added to the formulation.

Suitable antifoaming agents are for example antifoaming agents based on silicon or magnesium stearate.

Suitable preservatives are for example dichlorophen and benzyl alcohol hemiformal

Suitable thickeners are compounds which confer a pseudoplastic flow behavior to the formulation, i.e. high viscosity at rest and low viscosity in the agitated stage. Mention may be made, in this context, for example, of commercial thickeners based on polysaccharides, such as Xanthan Gum® (Kelzan® from Kelco), Rhodopol®23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations, i.e. the compositions according to the invention, comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of the present invention can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

The following are examples of formulations:

1. Products for dilution with water. For seed treatment purposes, such products may be applied to the seed diluted or undiluted.

A) Water-soluble concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

B) Dispersible concentrates (DC)

20 parts by weight of the active compound is dissolved in 70 parts by weight of cyclohexanone with addition of 10 parts by weight of a dispersant, for example polyvinylpyrrolidone. Dilution with water gives a dispersion, whereby a formulation with 20% (w/w) of active compounds is obtained.

C) Emulsifiable concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

F) Water-dispersible granules and water-soluble granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

G) Water-dispersible powders and water-soluble powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

I) Dustable powders (DP, DS)

5 parts by weight of the active compound are ground finely and mixed intimately with 95 parts by weight of finely divided kaolin. This gives a dustable product having 5% (w/w) of active compound.

J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

K) ULV solutions (UL)

10 parts by weight of the active compound is dissolved in 90 parts by weight of an organic solvent, for example xylene. This gives a product having 10% (w/w) of active compound, which is applied undiluted for foliar use.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

In the methods and uses of this invention, the compounds according to the invention may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following categorized list M of pesticides represents insecticidal mixture partners, which are, whenever possible, classified according to the Insecticide Resistance Action Committee (IRAC), and together with which the compounds according to the present invention may be used. The combined use of the compounds of the present invention with the following pesticides may result in potential synergistic effects. The following examples of insecticidal mixing partners are provided with the intention to illustrate the possible combinations, but not to impose any limitation to the obtainable mixtures:

M.1 Acetylcholine esterase (AChE) inhibitors from the class of

M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of

M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl)salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as:

M.2A cyclodiene organochlorine compounds, as for example endosulfan or chlordane; or

M.2B fiproles (phenylpyrazoles), as for example ethiprole, fipronil, flufiprole, pyrafluprole and pyriprole;

M.3 Sodium channel modulators from the class of

M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin,metofluthrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or

M.3B sodium channel modulators such as DDT or methoxychlor;

M.4 Nicotinic acetylcholine receptor agonists (nAChR) from the class of

M.4A neonicotinoids, for example acteamiprid, chlothianidin, dinotefuran, imidacloprid, nitenpyram, thiacloprid and thiamethoxam; or

M.4B nicotine.

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as

M.7A juvenile hormone analogues as hydroprene, kinoprene and methoprene; or others as

M.7B fenoxycarb, or

M.7C pyriproxyfen;

M.8 miscellaneous non-specific (multi-site) inhibitors, for example

M.8A alkyl halides as methyl bromide and other alkyl halides, or

M.8B chloropicrin, or

M.8C sulfuryl fluoride, or

M.8D borax, or

M.8E tartar emetic;

M.9 Selective homopteran feeding blockers, for example

M.9B pymetrozine, or

M.9C flonicamid;

M.10 Mite growth inhibitors, for example

M.10A clofentezine, hexythiazox and diflovidazin, or

M.10B etoxazole;

M.11 Microbial disruptors of insect midgut membranes, for example bacillus thuringiensis or bacillus sphaericus and the insecticidal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase, for example

M.12A diafenthiuron, or

M.12B organotin miticides such as azocyclotin, cyhexatin or fenbutatin oxide, or

M.12C propargite, or

M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluoron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example

M.20A hydramethylnon, or

M.20B acequinocyl, or

M.20C fluacrypyrim;

M.21 Mitochondrial complex I electron transport inhibitors, for example

M.21A METI acaricides and insecticides such as fenazaquin, fenpyroximate, pyrimidifen, pyridaben, tebufenpyrad or tolfenpyrad, or

M.21B rotenone;

M.22 Voltage-dependent sodium channel blockers, for example

M.22A indoxacarb, or

M.22B metaflumizone;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example

M.24A phosphine such as aluminium phosphide, calcium phosphide, phosphine or zinc phosphide, or

M.24B cyanide.

M.25 Mitochondrial complex II electron transport inhibitors, such as beta-ketonitrile derivatives, for example cyenopyrafen or cyflumetofen;

M.28 Ryanodine receptor-modulators from the class of diamides, as for example flubendiamide, chloranthraniliprole (Rynaxypyr®), cyanthraniliprole (Cyazypyr®), or the phthalamide compounds

M.28.1: (R)-3-chloro-N1-{2-methyl-4-[1,2,2,2-tetrafluoro-1-(trifluoromethyl)ethyl]phenyl}-N2-(1-methyl-2-methylsulfonylethyl)phthalamide and

M.28.2: (S)-3-chloro-N1-{2-methyl-4-[1,2,2,2-tetrafluoro-1-(trifluoromethyl)ethyl]phenyl}-N2-(1-methyl-2-methylsulfonylethyl)phthalamide, or the compound

M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide, or the compound

M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate;

M.X insecticidal active compounds of unknown or uncertain mode of action, as for example azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, or the compound

M.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound

M.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound

M.X.3: 11-(4-chloro-2,6-dimethylphenyl)-12-hydroxy-1,4-dioxa-9-azadispiro[4.2.4.2]-tetradec-11-en-10-one, or the compound

M.X.4: 3-(4′-fluoro-2,4-dimethylbiphenyl-3-yl)-4-hydroxy-8-oxa-1-azaspiro[4.5]dec-3-en-2-one, or the compound

M.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, I-1582).

The commercially available compounds of the group M listed above may be found in The Pesticide Manual, 15th Edition, C. D. S. Tomlin, British Crop Protection Council (2011) among other publications.

The phthalamides M.28.1 and M.28.2 are both known from WO 2007/101540. The anthranilamide M.28.3 has been described in WO2005/077943. The hydrazide compound M.28.4 has been described in WO 2007/043677. The quinoline derivative flometoquin is shown in WO2006/013896. The aminofuranone compounds flupyradifurone is known from WO 2007/115644. The sulfoximine compound sulfoxaflor is known from WO2007/149134. The isoxazoline compound M.X.1 has been described in WO2005/085216. The pyripyropene derivative M.X.2 has been described in WO 2006/129714. The spiroketal-substituted cyclic ketoenol derivative M.X.3 is known from WO2006/089633 and the biphenyl-substituted spirocyclic ketoenol derivative M.X.4 from WO2008/067911. Finally triazoylphenylsulfide like M.X.5 have been described in WO2006/043635 and biological control agents on basis of bacillus firmus in WO2009/124707.

The following list F of active substances, in conjunction with which the compounds according to the invention can be used, is intended to illustrate the possible combinations but does not limit them:

F.I) Respiration Inhibitors

F.I-1) Inhibitors of complex III at Qo site (e.g. strobilurins)

strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methyl-acetamide;
oxazolidinediones and imidazolinones: famoxadone, fenamidone;

F.I-2) Inhibitors of complex II (e.g. carboxamides):

carboxanilides: benodanil, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide, N-(4′-trifluoromethylthiobiphenyl-2-yl)-3 difluoromethyl-1-methyl-1H pyrazole-4-carboxamide and N-(2-(1,3,3-trimethyl-butyl)-phenyl)-1,3-dimethyl-5 fluoro-1H-pyrazole-4 carboxamide;

F.I-3) Inhibitors of complex III at Qi site: cyazofamid, amisulbrom;

F.I-4) Other respiration inhibitors (complex I, uncouplers)

diflumetorim; tecnazen; ferimzone; ametoctradin; silthiofam;
nitrophenyl derivates: binapacryl, dinobuton, dinocap, fluazinam, nitrthal-isopropyl,
organometal compounds: fentin salts, such as fentin-acetate, fentin chloride or fentin hydroxide;

F.II) Sterol biosynthesis inhibitors (SBI fungicides)

F.II-1) C₁₄ demethylase inhibitors (DMI fungicides, e.g. triazoles, imidazoles)

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole;
imidazoles: imazalil, pefurazoate, oxpoconazole, prochloraz, triflumizole;
pyrimidines, pyridines and piperazines: fenarimol, nuarimol, pyrifenox, triforine;

F.II-2) Deltal4-reductase inhitors (Amines, e.g. morpholines, piperidines)

morpholines: aldimorph, dodemorph, dodemorph-acetate, fenpropimorph, tridemorph;
piperidines: fenpropidin, piperalin;
spiroketalamines: spiroxamine;

F.II-3) Inhibitors of 3-keto reductase: hydroxyanilides: fenhexamid;

F.III) Nucleic acid synthesis inhibitors

F.III-1) RNA, DNA synthesis

phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;

F.III-2) DNA topisomerase inhibitors: oxolinic acid;

F.III-3) Nucleotide metabolism (e.g. adenosin-deaminase)

hydroxy (2-amino)-pyrimidines: bupirimate;

F.IV) Inhibitors of cell division and or cytoskeleton

F.IV-1) Tubulin inhibitors: benzimidazoles and thiophanates: benomyl, carbendazim, fuberidazole, thiabendazole, thiophanate-methyl;

triazolopyrimidines: 5-chloro-7 (4-methylpiperidin-1-yl)-6-(2,4,6-trifluorophenyl)-[1,2,4]triazolo[1,5 a]pyrimidine

F.IV-2) Other cell division inhibitors

benzamides and phenyl acetamides: diethofencarb, ethaboxam, pencycuron, fluopicolide, zoxamide;

F.IV-3) Actin inhibitors: benzophenones: metrafenone;

F.V) Inhibitors of amino acid and protein synthesis

F.V-1) Mmethionine synthesis inhibitors (anilino-pyrimidines)

anilino-pyrimidines: cyprodinil, mepanipyrim, nitrapyrin, pyrimethanil;

F.V-2) Protein synthesis inhibitors (anilino-pyrimidines)

antibiotics: blasticidin-S, kasugamycin, kasugamycin hydrochloride-hydrate, mildiomycin, streptomycin, oxytetracyclin, polyoxine, validamycin A;

F.VI) Signal transduction inhibitors

F.VI-1) MAP/Histidine kinase inhibitors (e.g. anilino-pyrimidines)

dicarboximides: fluoroimid, iprodione, procymidone, vinclozolin;
phenylpyrroles: fenpiclonil, fludioxonil;

F.VI-2) G protein inhibitors: quinolines: quinoxyfen;

F.VII) Lipid and membrane synthesis inhibitors

F.VII-1) Phospholipid biosynthesis inhibitors

organophosphorus compounds: edifenphos, iprobenfos, pyrazophos;
dithiolanes: isoprothiolane;

F.VII-2) Lipid peroxidation

aromatic hydrocarbons: dicloran, quintozene, tecnazene, tolclofos-methyl, biphenyl, chloroneb, etridiazole;

F.VII-3) Carboxyl acid amides (CAA fungicides)

cinnamic or mandelic acid amides: dimethomorph, flumorph, mandiproamid, pyrimorph;
valinamide carbamates: benthiavalicarb, iprovalicarb, pyribencarb, valifenalate and N-(1-(1-(4-cyano-phenyl)ethanesulfonyl)-but-2-yl)carbamic acid-(4-fluorophenyl)ester;

F.VII-4) Compounds affecting cell membrane permeability and fatty acides carbamates: propamocarb, propamocarb-hydrochlorid

F.VIII) Inhibitors with Multi Site Action

F.VIII-1) Inorganic active substances: Bordeaux mixture, copper acetate, copper hydroxide, copper oxychloride, basic copper sulfate, sulfur;

F.VIII-2) Thio- and dithiocarbamates: ferbam, mancozeb, maneb, metam, methasulphocarb, metiram, propineb, thiram, zineb, ziram;

F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;

F.VIII-4) Guanidines: guanidine, dodine, dodine free base, guazatine, guazatine-acetate, iminoctadine, iminoctadine-triacetate, iminoctadine-tris(albesilate);

F.VIII-5) Ahtraquinones: dithianon;

F.IX) Cell wall synthesis inhibitors

F.IX-1) Inhibitors of glucan synthesis: validamycin, polyoxin B;

F.IX-2) Melanin synthesis inhibitors: pyroquilon, tricyclazole, carpropamide, dicyclomet, fenoxanil;

F.X) Plant defence inducers

F.X-1) Salicylic acid pathway: acibenzolar-S-methyl;

F.X-2) Others: probenazole, isotianil, tiadinil, prohexadione-calcium;

phosphonates: fosetyl, fosetyl-aluminum, phosphorous acid and its salts;

F.XI) Unknown mode of action:

bronopol, chinomethionat, cyflufenamid, cymoxanil, dazomet, debacarb, diclomezine, difenzoquat, difenzoquat-methylsulfate, diphenylamin, flumetover, flusulfamide, flutianil, methasulfocarb, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;

F.XI) Growth regulators:

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;

F.XII) Biological control agents

antifungal biocontrol agents: Bacillus substilis strain with NRRL No. B-21661 (e.g. RHAPSODY®, SERENADE® MAX and SERENADE® ASO from AgraQuest, Inc., USA.), Bacillus pumilus strain with NRRL No. B-30087 (e.g. SONATA® and BALLAD® Plus from AgraQuest, Inc., USA), Ulocladium oudemansii (e.g. the product BOTRY-ZEN from BotriZen Ltd., New Zealand), Chitosan (e.g. ARMOUR-ZEN from BotriZen Ltd., New Zealand).

The invertebrate pest (also referred to as “animal pest”), i.e. the insects, arachnids and nematodes, the plant, soil or water in which the plant is growing or may grow can be contacted with the compounds of the present invention or composition(s) comprising them by any application method known in the art. As such, “contacting” includes both direct contact (applying the compounds/compositions directly on the invertebrate pest or plant—typically to the foliage, stem or roots of the plant) and indirect contact (applying the compounds/compositions to the locus of the invertebrate pest or plant).

The compounds of the present invention or the pesticidal compositions comprising them may be used to protect growing plants and crops from attack or infestation by animal pests, especially insects, acaridae or arachnids by contacting the plant/crop with a pesticidally effective amount of compounds of the present invention. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

Moreover, invertebrate pests may be controlled by contacting the target pest, its food supply, habitat, breeding ground or its locus with a pesticidally effective amount of compounds of the present invention. As such, the application may be carried out before or after the infection of the locus, growing crops, or harvested crops by the pest.

The compounds of the present invention can also be applied preventively to places at which occurrence of the pests is expected.

The compounds of the present invention may be also used to protect growing plants from attack or infestation by pests by contacting the plant with a pesticidally effective amount of compounds of the present invention. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 5 g to 500 g per hectare, more desirably from 5 g to 200 g per hectare.

The compounds of the present invention are effective through both contact (via soil, glass, wall, bed net, carpet, plant parts or animal parts), and ingestion (bait, or plant part).

The compounds of the present invention may also be applied against non-crop insect pests, such as ants, termites, wasps, flies, mosquitos, crickets, or cockroaches. For use against said non-crop pests, compounds of the present invention are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active ingredient.

Formulations of compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, ° leyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used. For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-H-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of the present invention and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of the present invention are also suitable for the treatment of plant propagation material, especially seeds, in order to protect them from insect pest, in particular from soil-living insect pests and the resulting plant's roots and shoots against soil pests and foliar insects.

The compounds of the present invention are particularly useful for the protection of the seed from soil pests and the resulting plant's roots and shoots against soil pests and foliar insects. The protection of the resulting plant's roots and shoots is preferred. More preferred is the protection of resulting plant's roots and shoots from chewing and biting insects, wherein the protection from Lepidoptera and Coleoptera is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the present invention, including a salt thereof. Particularly preferred is a method, wherein the plant's roots and shoots are protected, more preferably a method, wherein the plants roots and shoots are protected form chewing and biting insects, most preferably a method, wherein the plants roots and shoots are protected from Lepidoptera and Coleoptera.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting. The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A 242 236, EP-A 242 246) (WO 92/00377) (EP-A 257 993, U.S. Pat. No. 5,013,659) or in transgenic crop plants, for example cotton, with the capability of producing Bacillus thuringiensis toxins (Bt toxins) which make the plants resistant to certain pests (EP-A 142 924, EP-A 193 259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

D Emulsions (EW, EO, ES)

E Suspensions (SC, OD, FS)

F Water-dispersible granules and water-soluble granules (WG, SG)

G Water-dispersible powders and water-soluble powders (WP, SP, WS)

H Gel-Formulations (GF)

I Dustable powders (DP, DS)

Conventional seed treatment formulations include for example flowable concentrates SC, solutions LS, powders for dry treatment DS, water dispersible powders for slurry treatment WG, water-soluble powders SP and emulsion ES and EC and gel formulation GF. These formulations can be applied to the seed diluted or undiluted. Application to the seeds is carried out before sowing, either directly on the seeds or after having pregerminated the latter.

In a preferred embodiment a SC formulation is used for seed treatment. Typcially, a SC formulation may comprise 1-800 g/l of active ingredient, 1-200 g/l surfactant, 0 to 200 g/l antifreezing agent, 0 to 400 g/l of binder, 0 to 200 g/l of a pigment and up to 1 liter of a solvent, preferably water.

Especially preferred SC formulations of compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a Gelling Agent is Carrageen (Satiagel®)

In the treatment of seed, the application rates of the compounds of the present invention are generally from 0.1 g to 10 kg per 100 kg of seed, preferably from 0.5 g to 5 kg per 100 kg of seed, more preferably from 1 g to 1000 g per 100 kg of seed and in particular from 1 g to 200 g per 100 kg of seed.

The invention therefore also relates to seed comprising a compound of the present invention, including an agriculturally useful salt of it, as defined herein. The amount of the compound of the present invention, including an agriculturally useful salt thereof will in general vary from 0.1 g to 10 kg per 100 kg of seed, preferably from 0.5 g to 5 kg per 100 kg of seed, in particular from 1 g to 1000 g per 100 kg of seed. For specific crops such as lettuce the rate can be higher.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of the present invention into contact with each other, and “seed dressing” to methods of seed treatment which provide the seeds with an amount of the compounds of the present invention, i.e. which generate a seed comprising a compound of the present invention. In principle, the treatment can be applied to the seed at any time from the harvest of the seed to the sowing of the seed. The seed can be treated immediately before, or during, the planting of the seed, for example using the “planters box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of the present invention, including their stereoisomers, veterinarily acceptable salts or N-oxides, are in particular also suitable for being used for combating parasites in and on animals.

An object of the present invention is therfore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions comprising a parasiticidally effective amount of compounds of the present invention, including their stereoisomers, veterinarily acceptable salts or N-oxides, and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of the present invention, including its stereoisomers, veterinarily acceptable salts or N-oxides, or a composition comprising it.

The invention also provides a process for the preparation of a composition for treating, controlling, preventing or protecting animals against infestation or infection by parasites which comprises a parasiticidally effective amount of a compound of the present invention, including its stereoisomers, veterinarily acceptable salts or N-oxides, or a composition comprising it. Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula (I) and their stereoisomers, veterinarily acceptable salts, tautomers and N-oxides, are suitable for combating endo- and ectoparasites in and on animals.

The compounds of the present invention, especially compounds of formula (I) and their stereoisomers, veterinarily acceptable salts, tautomers and N-oxides, and compositions comprising them are preferably used for controlling and preventing infestations of and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of the present invention, including their stereoisomers, veterinarily acceptable salts or N-oxides, and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention, including their stereoisomers, veterinarily acceptable salts or N-oxides, and compositions comprising them are suitable for systemic and/or non-systemic control of ecto- and/or endoparasites. They are active against all or some stages of development.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus, cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp.,Knemidocoptes spp., Cytodites spp., and Laminosioptes spp,

bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

roundworms Nematoda:

wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,

thorny headed worms (Acanthocephala), e.g. Acanthocephalus spp., Macracanthorhynchus hirudinaceus and Oncicola spp,

Planarians (Plathelminthes):

flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The present invention relates to the therapeutic and the non-therapeutic use of compounds of the present invention and compositions comprising them for controlling and/or combating parasites in and/or on animals. The compounds of the present invention and compositions comprising them may be used to protect the animals from attack or infestation by parasites by contacting them with a parasiticidally effective amount of compounds of the present invention and compositions containing them.

The compounds of the present invention and compositions comprising them can be effective through both contact (via soil, glass, wall, bed net, carpet, blankets or animal parts) and ingestion (e.g. baits). As such, “contacting” includes both direct contact (applying the pesticidal mixtures/compositions containing the compounds of the present invention directly on the parasite, which may include an indirect contact at its locus-P, and optionally also administrating the pesticidal mixtures/composition directly on the animal to be protected) and indirect contact (applying the compounds/compositions to the locus of the parasite). The contact of the parasite through application to its locus is an example of a non-therapeutic use of compounds of the present invention. “Locus-P” as used above means the habitat, food supply, breeding ground, area, material or environment in which a parasite is growing or may grow outside of the animal.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions of the present invention. A parasiticidally effective amount of the compositions will also vary according to the prevailing conditions such as desired parasiticidal effect and duration, target species, mode of application, and the like.

The compounds of the present invention can also be applied preventively to places at which occurrence of the pests or parasites are expected.

Administration can be carried out both prophylactically and therapeutically. Administration of the active compounds is carried out directly or in the form of suitable preparations, orally, topically/dermally or parenterally.

### EXAMPLES

The present invention is now illustrated in further details by the following examples, without imposing any limitation thereto.

The compounds can be characterized e.g. by coupled High Performance Liquid Chromatography/mass spectrometry (HPLC/MS), by ¹H-NMR and/or by their melting points. The following analytical procedures were employed:

Analytical HPLC column: RP-18 column Chromolith Speed ROD from Merck KgaA (Germany).

Elution: acetonitrile+0.1% trifluoroacetic acid (TFA)/water+0.1% trifluoroacetic acid (TFA) in a ratio of from 5:95 to 95:5 in 5 minutes at 40° C.

Analytical UPLC column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 mL/min in 1.50 minutes at 60° C.

MS-method: ESI positive.

¹H-NMR: The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singulett.

Abbreviations used are: h for hour(s), min for minute(s) and room temperature for 20-25° C.

### A. Synthesis Examples

### Starting Materials

### Preparation Examples P.1 to P.10

Substituted 1H-benzo[d][1,3]oxazine-2,4-diones can be prepared according to WO 2007/43677 or by the following protocol of example P.1:

### Example P.1

### 6-chloro-8-methyl-1H-benzo[d][1,3]oxazine-2,4-dione

To a solution of 2-amino-3-methyl-5-chlorobenzoic acid (76.5 g, 0.41 mol) in dioxane (428 mL) was added diphosgene (59.7 mL, 97.8 g, 198 mmol, 1.20 equiv.) keeping the internal temperature below 28° C. The reaction was stirred at ambient temperature for 3 h, purged with nitrogen and then cooled to 0° C. The resulting precipitate was collected by filtration, washed with diisopropyl ether and dried in vacuum to obtain the title compound (87.10 g, 100%). Characterization by ¹H-NMR (400 MHz, DMSO-d₆): δ [delta]=2.32 (s, 3H), 7.66 (s, 1H), 7.75 (s, 1H), 11.19 (s, 1H).

### Example P.2

S-cyclopropylmethyl-5-ethyl sulfide was prepared according to Anderson, B.; Journal of Organic Chemistry (1962), 27, 2720-2724.

S,S-dicyclopropylmethyl sulfide and S-dicyclopropylmethyl-5-isopropyl sulfide can be prepared by analogy to S-cyclopropylmethyl-S-ethyl sulfide.

### Example P.3

S-cyclopropylmethyl-5-ethyl sulfinium sulfate (a compound of formula (III′) with L=CH₂, G=cyclopropyl, R⁵=ethyl, k=0 and A⁻=½ SO₄²⁻)

To a solution of sodium methylate (7.75 g of a 30% solution in methanol, 43.0 mmol, 1.10 equiv.) in methanol (200 mL) was added S-cyclopropylmethyl-S-ethyl sulfide (5.00 g, 7.6 mmol, 1.10 equiv.) at −5-0° C. To this mixture was added hydroxylamine-O-sulfonic acid (4.422 g, 39.10 mmol) at −20° C. and the reaction was allowed to reach room temperature slowly. After stirring at room temperature over night, all solids were removed by filtration. The filtrate was concentrated in vacuo and the residue was triturated with acetonitrile (50 mL) to yield the title compound (6.10 g, 43%).

The following compounds were prepared by analogy to example P.3:

### Example P.4

### S,S-dicyclopropylmethyl sulfinium sulfate (a compound of formula (III′) with L=CH₂, G=cyclopropyl, R⁵═CH₂-cyclopropyl, k=0 and A⁻=½ SO₄²⁻)

### Example P.5

S-cyclopropylmethyl-5-isopropyl sulfinium sulfate (a compound of formula (III′) with L=CH₂, G=cyclopropyl, R⁵=isopropyl, k=0 and A⁻=½ SO₄²⁻)

The following compound was prepared by analogy to example P.1:

### Example P.6

### 6-cyano-8-methyl-1H-benzo[d][1,3]oxazine-2,4-dione

### Example P.7

### 2-(3-Chloropyridin-2-yl)-5-trifluoromethyl-2H-pyrazole-3-carbonyl chloride (a compound of formula (VIII) with Z═Cl and R⁴═Cl)

In a reaction vessel equipped with a thermometer, septum, nitrogen inlet and stirring bar, 1-(3-chloro-2-pyridyl)-3-trifloromethyl-1H-pyrazole (10.0 g, 40.4 mmol) was dissolved in dry dimethoxyethane (50 mL). By means of a syringe, a 2 M solution of isopropyl magnesium chloride in tetrahydrofuran (40.4 mL, 80.8 mmol, 2.0 equiv.) was added dropwise with stirring, while cooling the vessel with an ice bath and keeping the internal temperature at about 5° C. The mixture was stirred for further 2 hours at 5° C. Then the ice-bath was removed and carbon dioxide was bubbled through the mixture causing an increase of the temperature up to 28° C. After the exothermic reaction has ceased, the mixture was cooled and all volatiles were removed by evaporation. The residue was taken up in dichloromethane (50 mL) and one drop of dry DMF was added. To this mixture thionyl chloride (14.41 g, 121.2 mmol, 3.0 equiv.) was added and the resulting mixture was heated to reflux for 3 hours. After cooling, the resulting precipitate was removed by filtration and the mother liquid was concentrated in vacuum to obtain the title compound (13.0 g, yield 100%, purity>85%) which was used in the next step without further purification.

Characterization by ¹H-NMR (400 MHz, CDCl₃): δ [delta]=7.43-7.54 (m, 2H), 7.93 (d, 1H), 8.52 (m, 1H).

### Example P.8

### 2-amino-5-chloro-N-(cyclopropylmethyl-ethyl-λ⁴-sulfanylidene)-3-methyl-benzamide (a compound of formula (VII) with L=CH₂, G=cyclopropyl, R⁵=ethyl, R³═H, R²═Cl, R¹=methyl and k=0)

To a solution of 6-chloro-8-methyl-1H-3,1-benzoxazine-2,4-dione (0.600 g, 2.55 mmol) in dichloromethane (20 mL) was added S-cyclopropylmethyl-S-ethyl sulfinium sulfate as obtained from example P.3 (0.644 g, 1.79 mmol, 0.70 equiv.) and potassium tert-butylate (0.315 g, 2.81 mmol, 1.10 equiv.) at room temperature. The mixture was stirred for 2.5 h, upon which water was added and the layers were separated. The aqueous layer was extracted with dichloromethane, combined organic layers were dried over sodium sulfate and concentrated in vacuo. The residue was purified by flash-chromatography on silica gel to yield the title compound (0.74 g, 97%). Characterization by UPLC-MS: 1.197 min, M=299.1

By the methods described in example P.8 the following compounds have been prepared:

### Example P.9

### 2-amino-5-chloro-N-(cyclopropylmethyl-2-propyl-λ⁴-sulfanylidene)-3-methyl-benzamide

### Example P.10

### 2-amino-5-bromo-N-(bis-cyclopropylmethyl-λ⁴-sulfanylidene)-3-methyl-benzamide

### Preparation Examples

### Examples 1 to 25

### Example 1

### 2-(3-chloro-2-pyridyl)-N-[2-methyl-4-chloro-6-[(cyclopropylmethyl-ethyl-λ⁴-sulfanylidene)carbamoyl]phenyl]-5-(trifluoromethyl)pyrazole-3-carboxamide (a compound of formula (I) with L=CH₂, G=cyclopropyl, R⁵=ethyl, R⁴═Cl, R³═H, R²═Cl, R¹=methyl and k=0)

To a solution of 2-amino-5-chloro-N-(cyclopropylmethyl-ethyl-λ⁴-sulfanylidene)-3-methyl-benzamide as obtained from example P.8 (0.640 g, 2.14 mmol) in toluene (5 mL) was added potassium carbonate (0.325 g, 2.35 mmol, 1.10 equiv.) and the resulting mixture was heated to 60° C. To this mixture, a solution of 2-(3-chloro-2-pyridyl)-5-(trifluoromethyl)pyrazole-3-carbonyl chloride (0.664 g, 2.14 mmol, 1.00 equiv.) in toluene (5 mL) was added dropwise and the mixture was stirred for 2.5 h at this temperature. The mixture was cooled and concentrated in vacuum. Water was added and the mixture was extracted with ethyl acetate. Combined organic layers were washed with water and brine, dried over sodium sulfate, filtered and concentrated in vacuum. Flash-chromatography on silica gel yielded the title compound (0.18 g, 15%).

Characterization by ¹H-NMR (400 MHz, DMSO-d₆): δ [delta]=0.40 (m, 1H), 0.48 (m, 1H), 0.76 (m, 2H), 1.40 (t, 3H), 2.21 (s, 3H), 2.93 (m, 1H), 3.09 (m, 2H), 3.20 (m, 1H), 7.23 (s, 1H), 7.27 (s, 1H), 7.41 (m, 1H), 7.86 (m, 1H), 7.92 (m, 1H), 8.47 (m, 1H), 11.73 (br s, 1H).

By analogy to the procedure described in example 1 the compounds of examples 2 to 25 of the following formula, as described in the following table, have been prepared.

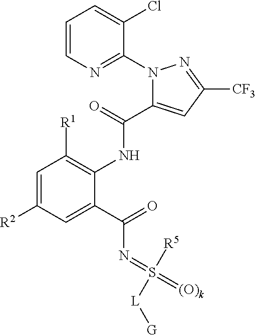

| | | | | | | | HPLC/ |
|---|---|---|---|---|---|---|---|
| | | | | | | | MS |
| Ex. | R¹ | R² | R⁵ | L | G | k | (Method) |
| 1 | CH₃ | Cl | CH₂CH₃ | CH₂ | cyclopropyl | 0 | 3.714 min, |
| | | | | | | | m/z = 572.0 |
| | | | | | | | (A) |
| 2 | CH₃ | Cl | CH(CH₃)₂ | CH₂ | cyclopropyl | 0 | 1.372 min; |
| | | | | | | | m/z = 586.1 |
| | | | | | | | (B) |
| 3 | CH₃ | Cl | CH₂- | CH₂ | cyclopropyl | 0 | 3.735 min; |
| | | | cyclopropyl | | | | m/z = 598.0 |
| | | | | | | | (A) |
| 4 | CH₃ | Cl | CH₂CH₃ | | cyclopropyl | 0 | 1.238 min; |
| | | | | | | | m/z = 558.0 |
| | | | | | | | (B) |
| 5 | CH₃ | Cl | CH(CH₃)₂ | | cyclopropyl | 0 | 1.257 min; |
| | | | | | | | m/z = 572.1 |
| | | | | | | | (B) |
| 6 | CH₃ | Cl | CH(CH₃)₂ | CH(CH₃) | cyclopropyl | 0 | 1.274 min; |
| | | | | | | | m/z = 532.1 |
| | | | | | | | (B) |
| 7 | CH₃ | Cl | CH₂-cyclobutyl | CH₂ | cyclobutyl | 0 | 1.429 min; |
| | | | | | | | m/z = 626.3 |
| | | | | | | | (B) |
| 8 | CH₃ | CN | CH₂-cyclobutyl | CH₂ | cyclobutyl | 0 | 1.374 min; |
| | | | | | | | m/z = 617.2 |
| | | | | | | | (B) |
| 9 | CH₃ | Cl | CH₂CH₃ | CH₂ | cyclobutyl | 0 | 1.327 min; |
| | | | | | | | m/z = 586.3 |
| | | | | | | | (B) |
| 10 | CH₃ | CN | CH₂CH₃ | CH₂ | cyclobutyl | 0 | 1.268 min; |
| | | | | | | | m/z = 577.4 |
| | | | | | | | (B) |
| 11 | CH₃ | Cl | CH₂Cl₂- | CH₂CH₂ | cyclopropyl | 0 | 1.422 min; |
| | | | cyclopropyl | | | | m/z = 626.4 |
| | | | | | | | (B) |
| 12 | CH₃ | CN | CH₂CH₂- | CH₂CH₂ | cyclopropyl | 0 | 1.376 min; |
| | | | cyclopropyl | | | | m/z = 617.5 |
| | | | | | | | (B) |
| 13 | CH₃ | Cl | CH(CH₃)₂ | CH₂CH₂ | cyclopropyl | 0 | 1.347 min; |
| | | | | | | | m/z = 600.3 |
| | | | | | | | (B) |
| 14 | CH₃ | CN | CH(CH₃)₂ | CH₂CH₂ | cyclopropyl | 0 | 1.313 min; |
| | | | | | | | m/z = 591.3 |
| | | | | | | | (B) |
| 15 | CH₃ | Cl | CH₂CH₃ | CH₂CH₂ | cyclopropyl | 0 | 1.311 min; |
| | | | | | | | m/z = 586.4 |
| | | | | | | | (B) |
| 16 | CH₃ | CN | CH₂CH₃ | CH₂CH₂ | cyclopropyl | 0 | 1.264 min; |
| | | | | | | | m/z = 577.4 |
| | | | | | | | (B) |
| 17 | CH₃ | Cl | CH(CH₃)₂ | CH₂ | cyclobutyl | 0 | 1.373 min; |
| | | | | | | | m/z = 600.3 |
| | | | | | | | (B) |
| 18 | CH₃ | Cl | CH₂- | CH₂ | cyclopentyl | 0 | 1.484 min; |
| | | | cyclopentyl | | | | m/z = 654.3 |
| | | | | | | | (B) |
| 19 | CH₃ | CN | CH₂- | CH₂ | cyclopentyl | 0 | 1.459 min; |
| | | | cyclopentyl | | | | m/z = 645.4 |
| | | | | | | | (B) |
| 20 | CH₃ | Cl | CH₂CH₃ | CH₂ | cyclopentyl | 0 | 1.372 min; |
| | | | | | | | m/z = 600.3 |
| | | | | | | | (B) |
| 21 | CH₃ | CN | CH₂CH₃ | CH₂ | cyclopentyl | 0 | 1.310 min; |
| | | | | | | | m/z = 591.4 |
| | | | | | | | (B) |
| 22 | CH₃ | Cl | CH(CH₃)₂ | CH₂ | cyclopentyl | 0 | 1.395 min; |
| | | | | | | | m/z = 616.0 |
| | | | | | | | (B) |
| 23 | CH₃ | CN | CH(CH₃)₂ | CH₂ | cyclobutyl | 0 | 1.306 min; |
| | | | | | | | m/z = 591.4 |
| | | | | | | | (B) |
| 24 | CH₃ | CN | CH(CH₃)₂ | CH₂ | cyclopentyl | 0 | 1.350 min; |
| | | | | | | | m/z = 605.5 |
| | | | | | | | (B) |
| 25 | CH₃ | Cl | CH₂CH₃ | CH₂ | cyclobutyl | 1 | 1.313 min; |
| | | | | | | | m/z = 602.3 |
| | | | | | | | (B) |

Method A: Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

Method B: Analytical UPLC column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 mL/min in 1.50 minutes at 60° C.

### B. Biological Examples

The activity of the compounds of formula I of the present invention could be demonstrated and evaluated in biological tests described in the following.

If not otherwise specified the test solutions are prepared as follow:

The active compound is dissolved at the desired concentration in a mixture of 1:1 (vol:vol) distilled water:acteon. The test solution is prepared at the day of use and in general at concentrations of 1000 ppm, 500 ppm, 300 ppm,100 ppm, 30 ppm and 5 ppm (wt/vol).

B.1 Cowpea Aphid (Aphis craccivora)

The active compound is dissolved at the desired concentration in a mixture of 1:1 (vol:vol) distilled water:acetone. Surfactant (Alkamuls® EL 620) is added at a rate of 0.1% (vol/vol). The test solution is prepared at the day of use.

Potted cowpea plants were colonized with approximately 50-100 aphids of various stages by manually transferring a leaf tissue cut from infested plant 24 hours before application. Plants were sprayed after the pest population has been recorded. Treated plants are maintained on light carts at about 28° C. Percent mortality was assessed after 72 hours

In this test, the compounds 1, 3, 4, 5, 6, 12, 14, 16, 21, 23 and 24, respectively, at 300 ppm showed a mortality of at least 75% in comparison with untreated controls.

### B.2 Diamond Back Moth (Plutella xylostella)

The active compound is dissolved at the desired concentration in a mixture of 1:1 (vol:vol) distilled water:aceteone. Surfactant (Alkamuls® EL 620) is added at a rate of 0.1% (vol/vol).The test solution is prepared at the day of use.

Leaves of cabbage were dipped in test solution and air-dried. Treated leaves were placed in petri dish eslined with moist filter paper and inoculated with ten 3rd instar larvae. Mortality was recorded 72 hours after treatment. Feeding damages were also recorded using a scale of 0-100%.

In this test, the compounds 1, 2, 3, 4, 5, 6, 7, 8, 9, 10, 11, 12, 13, 14, 15, 16, 17, 18, 19, 20, 21, 22, 23, 24 and 25, respectively, at 300 ppm showed a mortality of at least 75% in comparison with untreated controls.

### B.3 Orchid Thrips (Dichromothrips corbetti)

Dichromothrips corbetti adults used for bioassay were obtained from a colony maintained continuously under laboratory conditions. For testing purposes, the test compound is diluted in a 1:1 mixture of acetone:water (vol:vol), plus 0.01% vol/vol Alkamuls® EL 620 surfactant.

Thrips potency of each compound was evaluated by using a floral-immersion technique. Plastic petri dishes were used as test arenas. All petals of individual, intact orchid flowers were dipped into treatment solution and allowed to dry. Treated flowers were placed into individual petri dishes along with about 20 adult thrips. The petri dishes were then covered with lids. All test arenas were held under continuous light and a temperature of about 28° C. for duration of the assay. After 3 days, the numbers of live thrips were counted on each flower, and along inner walls of each petri dish. The percent mortality was recorded 72 hours after treatment.

In this test, the compounds 1, 2, 3, 4, 5, 6, 8, 9, 10, 11, 12, 13, 14, 15, 16, 17, 20, 21, 22, 23 and 24, respectively, at 300 ppm showed a mortality of at least 75% in comparison with untreated controls.

### B.4 Silverleaf Whitefly (Bemisia argentifolii)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compounds 2, 6, 7, 8, 9, 10, 11, 12, 13, 14, 15, 16 and 17, respectively, at 300 ppm showed a mortality of at least 75% in comparison with untreated controls.

### B.5 Southern Armyworm (Spodoptera eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds 1, 2, 3, 4, 5, 6, 8, 9, 10, 11, 12, 13, 14, 15, 16, 17, 20, 21, 22, 23 and 24, respectively, at 1 ppm showed a mortality of at least 75% in comparison with untreated controls.

### B.6 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, the compounds 1, 2, 3, 4, 5, 6, 8, 9, 10, 11, 12, 13, 14, 15, 16, 17, 20, 21, 22, 23 and 24, respectively, at 2500 ppm showed a mortality of at least 75% in comparison with untreated controls.

### B.7a Tobacco Budworm (Heliothis virescens) I

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants were grown 2 plants to a pot and selected for treatment at the cotyledon stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 budworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds 1, 2, 3, 4, 5, 6, 7, 8, 9, 10, 11, 12, 13, 14, 15, 16, 17, 18, 19, 20, 21, 22, 23, 24 and 25, respectively, at 2500 ppm showed a mortality of at least 75% in comparison with untreated controls.

### B.7b Tobacco Budworm (Heliothis virescens) I

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants were grown 2 plants to a pot and selected for treatment at the cotyledon stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 budworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds 1, 2, 3, 4, 5, 6, 9, 11, 13, 15 and 17, respectively, at 10 ppm showed a mortality of at least 75% in comparison with untreated controls.

### B.8 Boll Weevil (Anthonomus grandis)

For evaluating control of boll weevil (Anthonomus grandis) the test unit consisted of 24-well-microtiter plates containing an insect diet and 20-30 A. grandis eggs.

The compounds were formulated using a solution containing 75% v/v water and 25% v/v DMSO. Different concentrations of formulated compounds were sprayed onto the insect diet at 20 μl, using a custom built micro atomizer, at two replications.

After application, microtiter plates were incubated at about 23±1° C. and about 50±5% relative humidity for 5 days. Egg and larval mortality was then visually assessed.

In this test, the compounds 1, 2, 3, 4, 5, 6, 7, 8, 9, 10, 11, 12, 13, 14, 15, 16, 17, 18, 19, 20, 21, 22, 23 and 24, respectively, at 2500 ppm showed a mortality of at least 75% in comparison with untreated controls.

### B.9 Colorado Potato Beetle (Leptinotarsa decemlineata)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Eggplants were grown 2 plants to a pot and were selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. The treated foliage was then cut and removed from the pot and placed in a Petri dish lined with moistened filter pa-per. Five beetle larvae were introduced into each Petri dish and the dish was covered by a Petri dish lid. Petri dishes were maintained in a growth room at about 25° C. and about 20-40% relative humidity for 4 days, avoiding direct exposure to fluorescent light (24 hour photoperiod) to prevent trapping of heat inside the dishes. Mortality and reduced feeding were assessed 4 days after treatment, compared to untreated control plants.

In this test, the compounds 1, 2, 3, 4, 5, 6, 7, 8, 9, 10, 12, 13, 14, 15, 16, 17, 18, 19, 20, 21, 22, 23 and 24, respectively, at 10 ppm showed a mortality of at least 75% in comparison with untreated controls.

1-24. (canceled)

25. A compound of the general formula (I) 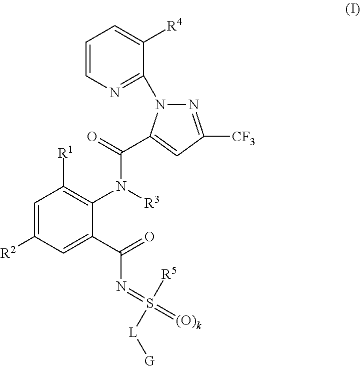
wherein
R¹ is selected from the group consisting of hydrogen, C₁-C₆-alkyl and C₃-C₈-cycloalkyl;
R² is selected from the group consisting of hydrogen, halogen and cyano;
R³ is selected from the group consisting of hydrogen, C₁-C₆-alkyl, C₁-C₆-haloalkyl, C₂-C₆-alkenyl, C₂-C₆-haloalkenyl, C₂-C₆-alkinyl, C₂-C₆-haloalkinyl, C₃-C₈-cycloalkyl, C₃-C₈-halocycloalkyl, C₁-C₄-alkoxy-C₁-C₄-alkyl, C₁-C₄-haloalkoxy-C₁-C₄-alkyl,
C(═O)R^{a}, C(═O)OR^{b} and C(═O)NR^{c}R^{d};
R⁴ is halogen or C₁-C₆-haloalkyl;
R⁵ is selected from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ is a 3-, 4-, 5-, 6- or 7-membered saturated, partially unsaturated or fully unsaturated heterocyclic ring containing 1, 2 or 3 heteroatoms or heteroatom groups selected from the group consisting of N, O, S, NO, SO and SO₂, as ring members, where the heterocyclic ring may be substituted by one or more radicals R^{f};
L is selected from the group consisting of CH₂, CH₂CH₂, CH(CH₃) and C(CH₃)₂.
C₁-C₈-alkanediyl, C₂-C₈-alkenediyl, C₂-C₈-alkynediyl and C₃-C₈-cycloalkanediyl, where one or more CH₂ groups of the aforementioned radicals may be replaced by a C═O group, and where the aliphatic and cycloaliphatic moieties of the aforementioned radicals may be unsubstituted, partially or fully halogenated and/or may carry 1 or 2 substituents selected from the group consisting of C₁-C₄ alkoxy, C₁-C₄ alkyl and C₁-C₄ haloalkyl;
G is selected from the group consisting of C₃-C₈-cycloalkyl, which is unsubstituted or carries 1 to 10 substituents R^{e};
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from the group consisting of C₁-C₄ alkoxy;
phenyl, benzyl, pyridyl and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from the group consisting of C₁-C₄-alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl; or
R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from the group consisting of N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from the group consisting of C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy and C₁-C₆-haloalkoxy; or
two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from the group consisting of C₁-C₄ alkoxy;
C₁-C₆-alkoxy, C₁-C₆-haloalkoxy, C₁-C₆-alkylthio, C₁-C₆-alkyl sul finyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
k is 0 or 1;
n is 0, 1 or 2;
or a stereoisomer, salt, tautomer or N-oxide thereof.

26. The compound according to claim 25, wherein R¹ is selected from the group consisting of methyl, ethyl, isopropyl, cyclopropyl, cyclobutyl, cyclopentyl and cyclohexyl.

27. The compound according to claim 25, wherein R² is selected from cyano, chlorine and bromine.

28. The compound according to claim 25, wherein R³ is hydrogen.

29. The compound according to claim 25, wherein R⁴ is selected from chlorine and bromine.

30. The compound according to claim 25, wherein R⁵ is selected from the group consisting of C₁-C₆-alkyl, C₃-C₆-cycloalkyl, C₂-C₆-alkenyl, C₂-C₆-alkynyl, wherein the aforementioned radicals may be substituted with 1 to 6 substituents R^{e}, and phenyl, which is unsubstituted or carries 1 to 4 radicals R^{f}, or R⁵ is a 5-, 6- or 7-membered saturated, partially unsaturated or fully unsaturated heterocyclic ring containing 1, 2 or 3 heteroatoms selected from the group consisting of N, O and S, as ring members, where the heterocyclic ring may be substituted by 1, 2 or 3 radicals selected from the group consisting of halogen, cyano, C₁-C₄-alkyl, C₁-C₄-alkoxy and C₁-C₄-haloalkyl.

31. The compound according to claim 25, wherein R⁵ is selected from the group consisting of CH₃, CH₂CH₃, CH═CH₂, CH₂CH₂CH₃, CH(CH₃)₂, CH₂CH₂CH₂CH₃, C(CH₃)₃, CH₂CH(CH₃)₂, CH(CH₃)CH₂CH₃, CH₂CH═CH₂, CH₂C≡CH, CH(CH₃)CH═CH₂, CHF₂, CH₂Cl, CH₂CH₂CN, CH₂CH₂Cl, cyclopropyl, cyclobutyl, cyclopentyl, cyclohexyl, cyclopropylmethyl, 1-cyclopropylethyl, cyclopentylmethyl, cyclohexylmethyl and phenyl; and in particular is CH₃, CH₂CH₃, CH(CH₃)₂ or cyclopropylmethyl.

32. The compound according to claim 25, wherein G is selected from C₃-C₇-cycloalkyl, which is unsubstituted or carries 1 to 4 substituents selected from the group consisting of halogen, cyano, C₁-C₆-alkyl and C₂-C₆-alkenyl.

33. The compound according to claim 25, wherein G is selected from C₃-C₇-cycloalkyl, which is unsubstituted or carries 1 to 4 substituents selected from the group consisting of halogen, cyano and C₁-C₄-alkyl.

34. The compound according to claim 25, wherein G is C₃-C₇-cycloalkyl.

35. The compound according to claim 25, wherein k is 0.

36. The compound according to claim 25, wherein the compound has the general formula (I-a) 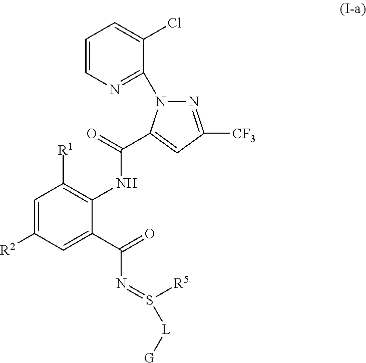
wherein R¹, R², R⁵, L and G are as defined in any of the preceding claims.

37. The compound according to claim 25, wherein
R¹ is methyl;
R² is Cl, Br or CN;
R³ is hydrogen;
R⁴ is Cl;
R⁵ is CH₃, CH₂CH₃, CH(CH₃)₂ or cyclopropylmethyl,
L is CH₂ or CH(CH₃);
G is cyclopropyl, cyclopentyl or cyclohexyl; and
k is 0.

38. An agricultural or veterinary composition comprising at least one compound as defined in claim 25, or a stereoisomer, agriculturally or veterinarily acceptable salt, tautomer or N-oxide thereof and at least one liquid and/or solid carrier.

39. A method for combating or controlling invertebrate pests, which method comprises contacting said pest or its food supply, habitat or breeding grounds with a pesticidally effective amount of at least one compound as defined in claim 25 or a stereoisomer, salt, tautomer or N-oxide thereof, except for a method performed on humans.

40. A method for protecting growing plants from attack or infestation by invertebrate pests, which method comprises contacting a plant, or soil or water in which the plant is growing or may grow, with a pesticidally effective amount of at least one compound as defined in claim 25 or a stereoisomer, salt, tautomer or N-oxide thereof.

41. A method for the protection of seeds from soil insects and of the seedlings' roots and shoots from invertebrate pests comprising contacting the seeds before sowing and/or after pregermination with at least one compound as defined in claim 25 or a stereoisomer, salt, tautomer or N-oxide thereof.

42. Seed treated with a compound as defined in claim 25 or a stereoisomer, salt, tautomer or N-oxide thereof in an amount of from 0.1 g to 10 kg per 100 kg of the plant propagation material.

43. A method for treating a non-human animal infested or infected by parasites or for preventing a non-human animal from getting infested or infected by parasites or for protecting a non-human animal against infestation or infection by parasites which comprises orally, topically or parenterally administering or applying to the non-human animal a parasiticidally effective amount of a compound as defined in claim 25 or a stereoisomer, veterinarily acceptable salt, tautomer or N-oxide thereof.
